SEQUENCE LISTING (27 sequences)

SEQ ID NO: 1 - DNA, 1145 bp, Helicobacter pylori
atgaaagaaaaaatcgctttaatcaccggggttaccgggcaagacgggagctatctggctgaatacttgctgaatttgggttatgaagtgcatgggttaaaaaggcgctcttctagcatcaacacttctaggatcgatcatttgtatgaagatttgcacagcgaacacaaaaggcgttttttcttgcactatggggatatgaccgatagctctaacctcatccatttgatgctaccactaagcccacagagatttataatttagccgcgcaaagccatgtgaaagtctcttttgaaaccccagaatacaccgctaacgctgatggtattggcacgctaaggattttagaggccatgcggattttgggcttagaaaagaaaacgcgcttttatcaagctagcacgagcgaattgtatggcgaagtcttagaaaccccacaaaatgaaaacaccccctttaacccacgaagcccctatgcggtcgctaaaatgtatgccttttacatcactaaaaattacagagaggcttataatttgtttgcggttaatggcatcctttttaaccatgagagcagggtaaggggcgaaacttttgtaacccgtaaaatcacacgagccgctagcgcgatagcgtataacttaacggattgcttgtatttagggaatttggacgctaaaagagactgggggcatgccaaagattatgtgaaaatgatgcatttgatgctccaagcacccaccccacaagattatgtaatcgctacagggaagaccacgagcgtgcgcgattttgtgaaaatgagctttgaatttatcggcattgatctagaatttcaaaatacagggattaaagaaatcggtttgattaaaagcgttgatgaaaaaagagcgaacgctttacaattaaacttaagccatttaaaaacgggcaaaatcgtggtgcgtatagacgagcactatttcaggcctactgaagtggatttgctcttaggcgatcccactggggctgagaaagagctgggctgggttagggaatacgatttaaaagagttggttaaggacatgttagaatacgatttaaaagaatgccagaaaaacctttacttgcaagatgggggctatactttaaggaatttttatgaatga

SEQ ID NO: 2 - DNA, 933 bp, Helicobacter pylori
atgaatgagatcattttaatcaccggcgcttatggcatggtggggcagaacacggcgttgtattttaaaaaaaacaagcctgatgttaccttactcacccctaaaaagagcgaattgtatttgttggataaagacaacgttcaagcctatttgaaagaatacaagcctacaggcattatccattgtgccgggagagtggggggcattgtggcaaacatgaacgatctttcaacttacatggttgagaatttactcatgggtttgtatcttttttctagcgctttagatttgggcgtgaaaaaagccattaatctagcgagctcttgcgcttatcctaaatacgcccctaaccctttaaaagagagcgatttattgaacggctctttagaaccaacgaatgaaggctacgctttagccaaactctctgtgatgaagtattgcgaatacgtgagcgctgaaaaaggcgttttttataaaactctagtgccttgtaacctttatggcgagtttgacaagtttgaagaaaagatagcgcacatgataccagggcttattgctaggatgcacaccgctaaattaaaaaatgaaaaaaattttgcgatgtggggcgatggcacggccagaagagagtatctaaacgctaaagatttagccagattcatcgctctcgcttatgagaatatcgctcaaatgcctagcgtgatgaatgtcggctctggagtggattacagcattgaagagtattacgaaaaagtcgctcaggttttagactataagggcgtgtttgtgaaagattcatccaaaccagtgggcatgcaacaaaagcttatggatatttccaaacaaaaggctttaaaatgggaattagaaatccctttagagcagggcatcaaagaagcttatgagtattatttgaagcttttagaggtttga

SEQ ID NO: 3 - DNA, 2388 bp, Murinae gen. sp.
atggagcagtcagagggagtcaattggactgtcattatcctgacatgccagtacaaggacagtgtccaggtctttcagagagagctggaggtaaggcagagacgggagcagattcctgcggggacgatgttactggctgtggaggatccccagactcgagtcggcagcggaggagccaccctcaacgcactgctggtggctgctgaacacttgagtgcccgagctggcttcactgtggtcacgtccgatgtcctgcactctgcctggatcctcatcttgcacatgggccgagacttccccttcgatgactgtggcagggccttcacttgcctccctgtggagaacccacaggcccctgtggaggccttggtatgcaacctggactgcctgttggatatcatgacccaccggctgggtccaggttccccaccaggtgtgtgggtctgcagcaccgacatgcttctgtctgttcctccaaaccctgggatcagttgggatggcttccggggagccagagtgatcgcctttcctgggagcctggcctatgcgttgaaccacggtgtctacctcactgactcacagggcttggttttggacatttactaccagggcactaaggcggagatacaacgttgtgtcggacctgatgggctggtaccattggtctccggggtcgtcttcttctctgtggagactgctgagcacctcctagccacccatgtgagcccaccgctggatgcctgcacctatatgggcttggactctggagcccagcctgtgcagctgtctctgtttttcgacatcctgctctgcatggctcggaatatgagcagggagaacttcctggctgggcggcccccggagttggggcaaggtgacatggatgtagcaagttacctgaagggagcccgggcccagctgtggagggagcttcgagatcagcccctcacaatggtgtatgtccctgacggcggctacagctacatgacgactgatgccaccgagttcctgcacagactcacgatgcctggagtagctgtggcacagattgttcactcccaggtggaggagccacagctgctagaggctacgtgctcggtggtcagctgcctgctcgagggccctgtgcacctggggcctcgaagtgtcctgcagcactgtcacctgaggggccccattcgcatcggcgctggctgctttgtgagtggtctggatacagcccactcggaggcactgcatggcctggagctccatgatgtcatcctgcagggacaccatgtgcggctgcatggctccctgagccgtgtatttactcttgctggccgtctggacagctgggaaaaaaagggggcaggcatgtatctcaacatgtcctggaatgagttcttcaagaagacaggcattcgagactgggacctgtgggaccaagatacacccccctcagatcgaggcctcctcactgcccgccttttccctgtgctccaccccacgagggccctggggccccaggatgtgctgtggatgctgcacccccgcaaacacagaggtgaggcccttcgggcctggcgagcctcctggcgtctgtcctgggagcagctgcaaccttgtgtggaccgggctgccacactggacttccgccgagatctgttcttctgccaggccttgcagaaggcaaggcatgtgttagaggcgcggcaggacctctgcctacgtccactgatccgggccgctgtcggggaaggttgctttgggcccctgctggccacacttgacaaggttgcagctggggcagaagatcctggcgtggcagcccgggctctggcttgtgtggccgatgtgctgggctgcatggcagagggtcgaggaggcttgcgcagtgggccagctgccaaccctgagtggattcagcctttctcatacttggagtgtggagacctgatgaggggtgtggaggcgcttgcccaggagagagagaagtggctgaccaggcctgccttgctggttcgagctgcccgccattacgagggggccgagcagatcctgatccgccaggctgtgatgacagcccggcacttcgtctccacccagcccatggagctgcccgcacccgggcagtgggtggtgactgagtgcccagcccgtgtggatttctctgggggctggagtgacacaccgcccattgcctatgagcttggtggagcagtgttgggcctggctgtgcgggtggatggccgccggcccatcggggccaaagcacgccgcatcccggagcctgagctctggctggcagtgggacctcggcaggatgagatgaccatgaggatagtgtgccggagcctggatgacctgcgggattactgccagcctcatgccccagggccttgctga

SEQ ID NO: 4 - DNA, 2469 bp, Homo sapiens
atggagcagccgaagggagttgattggacagtcatcatcctgacctgccagtacaaggacagtgtccaggtctttcagagagaactggaagtgcggcagaagcgggagcagatccctgctgggacgctgttactggccgtggaggacccagagaagcgtgtgggcagcggaggagccaccctcaacgccctgctggtggctgctgaacacctgagtgcccgggcaggcttcactgtggtcacatccgatgtcctgcactcggcctggatcctcattctgcacatgggtcgagacttcccctttgatgactgtggcagggctttcacctgcctccccgtggagaaccccgaggcccccgtggaagccttggtctgcaacctggactgcctgctggacatcatgacctatcggctgggcccgggctccccgccaggcgtgtgggtctgcagcaccgacatgctgctgtctgttcctgcaaatcctggtatcagctgggacagcttccggggagccagagtgatcgccctcccagggagcccggcctacgctcagaatcatggcgtctacctaactgacccccagggccttgttttggacatttactaccagggcactgaggcagagattcagcggtgtgtcaggcctgatgggcgggtgccactggtctctggggttgtcttcttctctgtggagactgccgagcgcctcctagccacccacgtgagcccgcccctggatgcctgcacctacctaggcttggactccggagcccggcctgtccagctgtctctgttttttgacattctccactgcatggctgagaacgtgaccagggaggacttcctggtggggaggcccccagagttggggcaaggcgatgcagatgtagcgggttatctgcagagcgcccgggcccagctgtggagggagcttcgcgatcagccccttaccatggcctatgtctccagcggcagctacagctacatgacctcctcagccagtgagttcctgctcagcctcacactccccggggctcctggggcccagattgtgcactcccaggtggaggagcagcagcttctggcggccgggagctctgtggtcagctgcctgctggagggccctgtccagctgggtcctgggagcgtcctgcagcactgccacctgcagggccccattcacataggcgctggctgcttggtgactggcctggatacagcccactccaaggccctgcatggccgggagctgcgtgaccttgtcctgcagggacaccacacgcggctacacggctccccgggccacgccttcaccctcgttggccgtctggacagctgggagagacagggggcaggcacatatctcaacgtgccctggagtgaattcttcaagaggacaggtgttcgagcctgggacctgtgggaccctgagacgctgcccgcagagtactgccttcccagcgcccgcctctttcctgtgctccacccctcgagggagctgggaccccaggacctgctgtggatgctggaccaccaggaggatgggggcgaggccctgcgagcctggcgggcctcctggcgcctgtcctgggagcagctgcagccgtgcctggatcgggctgccacgctggcctctcgccgggacctgttcttccgccaggccctgcataaggcgcggcacgtgctggaggcccggcaggacctcagcctgcgcccgctgatctgggctgctgtccgcgagggctgccccgggcccctgctggccacgctggaccaggttgcagctggggcaggagaccctggtgtggcggcacgggcactggcctgtgtggcggacgtcctgggctgcatggcagagggccgtgggggcttgcggagcgggccagctgccaaccctgagtggatgcggcccttctcatacctggagtgtggagacctggcagcgggcgtggaggcgcttgcccaggagagggacaagtggctaagcaggccagccttgctggtgcgagcggcccgccactatgagggggctggtcagatcctgatccgccaggctgtgatgtcagcccagcactttgtctccacagagcaggtggaactgccgggacctgggcagtgggtggtggctgagtgcccggcccgtgtggatttctctgggggctggagtgacacgccaccccttgcctatgagcttggcggggctgtgctgggcctggctgtgcgagtggacggccgccggcccatcggagccagggcacgccgcatcccggagcctgagctgtggctggcggtggggcctcggcaggatgagatgactgtgaagatagtgtgccggtgcctggctgacctgcgggactactgccagcctcatgccccaggtcaggcaccctgggacctctgcaggtggggctggccagctgggggctcggggcagaagctagagactgcaggccagtggggtctggcctga

SEQ ID NO: 5 - DNA, 3540 bp, Murinae gen. sp.
  misc_feature (2492)..(2492): unknown
ccctctagatgcatgctcgagcggccgccagtgtgatggatatctgcagaattcgcccttaagtcggccatggagactctccgcgaagccaccctgcggaaactgcgcagattttctgagctgagaggcaaacccgtggcagctggagaattctgggatgtggttgcaataacagcagctgatgaaaagcaggagctcgcttacaagcaacagttgtccgagaagctgaaaaaaaaggaattgcctcttggagttcaataccatgtttttccagatcctgctgggaccaaaattggaaatggaggatcgacactttgttcccttcagtgtttggaaagcctctgtggagacaaatggaattctctgaaggtcctgctaatccactctggtggctacagccaacgccttcccaatgcgagtgctttaggaaagatcttcactgccttaccacttggtgaacccttctatcagatgttggagttaaaactagccatgtacgtggatttcccctcaaacatgaggcctggagtcttggtcacctgtgcagataacatcgaactctacagtgttggggacagtgagtacattgcctttgaacagcctggctttactgccttagcccatccgtctagtctgggtgttgccactactcatggagtatttgtcttgcactctgacagttccctacaacatggtgaccttaagttaagacaatgctacaattccctccacaaccccaccattgaaaacatgccccaccttatttctgtgcaaagacaaggaagctttgctcaacaggacttgtctggaggtgacactgactgtcctccattgcacactgagtatgtctacacagatagcctgttttacatggatcgcaaatcagccaaaaagttacttgattctattaaaagtgaaggcccactgaactgtgaaatagatgccaatggagactttattcaggcactggggcctggagcaactgcagagtacaccaggaacacatctcatgtcactaaagaagagtcccagttgttggacatgaggcagaaaatattccacctcctcaagggaacaccactgaatgttgttgttcttaataactccagattttatcacattggaacactgcaagagtatctgcttcatttcacctctgatagtgcattaaagacgagagctgggcttacaatccatagctttcaagtgtctctccaagtgttcctgagcgctccagtggaacagcctgtgtcattcacagtatactgggattcaggatgctgtgtggcccctggctcagtggtagagtattctagattggggcctgaggtgtccatcggggaaaactgcattatcagcagttctgtcatagcaaaaactgttgtgccagcatattcttttttgtgttctttaagtgtgaagataaatggacacttaaaatattctactatggtgtttggcatgcaagacaacttgaagaacagtgttaaaacactggaagacataaaggcacttcagttctttggagtctgttttctgtcttgtttagacatttggaatcttaaagctacagagaaactattctctggaaataagatgaatctgagcctgtggactgcacgcattttccctgtctgttcatctctgagtgagtcggctacagcatcccttgggatgttaagcgctgtaaggaaccattcaccattcaacctaagtgactttaaccttttgtccatccaggaaatgcttgtctacaaagatgtacaagacatgctagcttatagggaacacatttttctagaaattagttcaaataaaaatcaatctgatttagagaaatcttaaattgatattttggccataaacaaaattgcaaatacaggcattttctatagacctctgacatttttgtttgttttaataaagtaatataataaaaattatgttaatataactgttgtagcttggtaatgagaatggtacaactgaccacttctgctagaagtacgttccaggactagagtcaggaaaggtcggctgttttagatgtttacaccatcttacaattgtgctctttggtaaagatccatttatgggcactgtttcattcacaaaataaatattctggtttatagggatgatttcttaacataacatatcttttaagcttttctatctcttttggaaatttggaccaataaaattctaggtgatattggaggatggtattgctcaacttctcatagtgagacaccccgtacaaaacatggttttaaatcttagaagaaatgtcatatttgagggttctttgaggcttgtcaaggtttaatttaagggggaaaaattgtttacccggaaggaaggaatggccccaattggcttggggcaccaaaaagggttaaaaaaactgtttccaaaaattggggtgggacccttttgggggcccaaacaatccttttggggggaacctaaggccattggaaaaccaaaantttccattattgtttcaaacaggagggaattcccagttagaagcaccaaaaaatattttaaattggggtcacacaggaatgggacttgattgaaggataccaggctccggaggtgaaaacctccggtttagaaaaagccatgggtcttttggaactaaagttaaagaagcttagacccccggggtggttctggtagcccacctcctttagtctatgaagtgctaaaaatgaggttgtgcctccatggtcttgccaaatgatataaaagatgtatggatgattttgttcttatacactagaacatgtgttgccatatcttataaactatgtctacttgatatattacactggtagctatgtacacacagaactcagttgtctgctcaggaggtggtagggatagttgagagccagtactcactcactatggaccttacttaatcctctcctagttaatccttctccaaatctcttaacttgacagtggacatttgccttgcatcattggtggtagtgatgctgtgaacaaacaataggcccaaagagaggaaattcaaataggcaatctgaagaactactcaaatcataaacaactgcagggaaatgaaatgggggaattcctggttatgcgtacctattatgaaataaacacattagtggaatgtccttaggttgaactgtaatagagttaaattttatcatacttgtgtttaaaataccttaagtacattgtaatatctgctgtggcaactttaattctgtgtaagttttcataaaaatatatgataaacaagatatctgtcaaaactcctttatattatttatataagaatatttgcctttttgaggtactagataataaagcaaagaatgtacgatactatatgacaattattggtaaagttacagagaattcaatggatgttaaatgttattaaatactcaagactaaagtcctatcaacgatgagaattatgatttcatgttccaagaaaaaaatatcattaataaagaataccatcacttccttgtaaaaaaaaataaaaaa

SEQ ID NO: 6 - DNA, 3461 bp, Murinae gen. sp.
ctcactatagggctcgagcggccgcccgggcaggtgtgggctcccggaagtcggccatggagactctccgggaagccaccctgcggaaactgcgcagattttcggagctgagaggcaaacctgtggcagctggagaattctgggatgtggttgcgataacagcagccgatgaaaagcaggagctcgcttacaagcagcagttgtcagaaaagctgagaagaaaggaattgcctcttggagttcaataccatgtttttcctgatcctgctgggaccaaaattggaaatggaggatcgacactttgttcccttcagtgcctaaaaagcctctatggagatgaatggaattctttcaaggtcctgttaattcactccggtggctacagtcaacgccttcccaatgcaagtgctttaggaaagatcttcacagccttaccacttggtgaacccatctatcagatgttggagttaaaactagccatgtacgtggatttcccctcacacatgaagcctggagtcttggtcacctgtgcagatgacattgaactgtacagtgttggggactgtcagtacattgcctttgaccagcctggctttactgccttagcccatccttccagtctggctgtaggcaccacacacggagtatttgtcttgcactctgccagttccttacaacatggtgaccttcagtacagacaatgccaccgtttcctccacaagcccaccattgaaaacatgcatcagtttaatgctgtgcaaagacaaggaagctttgctcaacaggacttccctggaggtgacaccgcgtgtcttccattgcacactgagtatgtctacacagatagcctgttttacatggaccacaaatcggccaaaaagttacttgatttctataaaaatgtaaaccaactgaactgtgaaatagatgcctatggtgactttctgcaggcactggggcctggagcaactgcagagtataccaggaacacatcacatgtcactaaagaagactcccagttgttggacatgaggcagaaaatattccacctcctcaaggggacaccactgaatgttgttgttcttaataactccagattttatcacattggaacaacacaagaatatctgcttcatttcacgtctgatagtacgttaaggtcaagagctaggcttacagtccatagctttcaagtgtctctccaagtatccctgaatcctccaatgaaacagcctgtatcattcacagtatactgggattcaggatgctgtgtggcacctggctcagttgtagagtattctagactggggcctgaggtgtccattggggaaaactgcattgtcagcagctctgtcctagcaaacactgctgtgccggcatattcttttgtgtgttctctaagtgtgaggacaaatggactcttggaatattctaccatggtgtttagtgtgcaggacaacttgaaaggcagtgttaaaaccctggaagatataaaggcacttcagttctttggagtctgtttcttgtcttgtttagacatctggaaccttaaagctacagagaaactgttctctggaagtaagaggaacctgagcctgtggactgcacggattttccctgtctgtccttctctgagtgagtcagttacagcatcccttgggatgttaagtgctgtaaggagccattcaccattcagcctaagcaactttaagctgatgtccatccaggaaatgcttgtctacaaagatgtacaagacatgctagcttatagggagcaaatttttctagaaattaattcaaataaaaaacaatctgatttagagaaatcttaaattgaacatattttgtctgtaaacaaaattgaaaatgcagatatttttctatagacctgactttttgtttagtaaatcaatataataaaagttatattaataaaaagcgttgtagcttggtaatgaataaggtatggctgagcacttcagctggaaggacattctgggactaaagtcaggaaagcaatgctgtttcagatgtttgcatcatcttaattttgctttttgggtgaaggtccatttacgggcactggtttggttcacagaataaatactcctgactatgggatgattttctaaacatcctttacagttttcctatcttctttggatttggccaataaagtctaggggatattgatgattatattaccttactctccaagtgagacaccatctacaatacattggtagaaaattcctaaaagaaattcatttttgagggtttcttcaaaatttgtcaagtttaagggacgaagctttacttgcatgtatgtttgggcaccgcatgctgctctgccaaaagctaaacagctgttctcacctgtggggtgcctttttgggatgaaccatttttgaggggtacctaagaccactgaaaataaaaaattcgcattgcgatttataacaggaaaattacagttaagaagtagcacaaagtaattttacagtttgggtcaccacagccttaggaactgtttttaaaggatggcagcatcaggaaggttgagaaccactggtctagaagaaggcattggatctgttggaatcagtatagatggttacaacctccatattgtttagtctgcaagtcaactttagtcctatgaaagtgcttataaaatgatgtttgtgcctcactgatcttgccaaaatgagattaaatgtacggtgtgtgttattcttaatacacttgaacatggggtgccatattttataaactacatcactgatctattgcactggaaaatgcatacacacagtatgcggttgtctgctcaggaggtggtaggaatagttgagagccagtgctcactcagcgtggacattactttatcctctcctaattaatccttcaacaagtctcttgacaatgagcatttgccttacatcgttttgtgtccatagtggtagtgatgctgtggacaagcaattggcccaaagacaggaaattccaataggaaatctgaggtaatactcagatcatacaggtagaattcctggttaaaggcatctattttgaaataaacacattagtggaatgtctttaggttgaacttgaatcgaggcgagacttatacttaaggaaactgtaatatctactgtgacatattacttatatgtaagttttcataaaaatatatgataaacaagatatctatcaaacccctctttattatttataatgaatatgtatttaaccttttgaggttctagacaataaagcaaagtatacgtgatgatatttgacagtgattgatttacagggatctcaaaggatgttgaatgctattaaatgttcaaaactaaagtcccatcaagtattagaattatgattgcatgttccaaaaaatctattattaataaagaataccatcacttccttgcaaaaaaaaaaaaaaaaaaaaa

SEQ ID NO: 7 - DNA, 1910 bp, Murinae gen. sp.
ggttgatgccaaagattgaaggggtagggtggggcagaagtgggaaggtccctggcttcctcaccttggtagatgaaacaaaagcatcaggctgagttgagcagtagctgtgatttcagggtgcctctgttggagaggctgctgtgatttgaaaacctcctttccctgggtgactaattccagaaagctctggatgaattgtattggtgagtgcctggccttgagaagtcccagctggggacgatggggattttggggtgtccccgaacctaaggtgacagggcctctcctttttcattctgcttcagggtgcaaccccgtctggaagctgcgggcctgggggtgtctggctggaggtacaacactcatggtaatctggctcttctggctgctgcgatcagttcctgggggtgccccagcgccccagcccacactcaccatccttatctggcactggcctttcaccaaccggtcgccagagctgtctagcgacacctgcactcgctatggcatggccagctgccacctgagtgctaaccggagtctgctagccagtgctgatgctgtggtcttccaccatcgtgagctgcagacccggcactctcgcctgcccctggaccagaggccgcacggacagccttgggtctgggccaccatggaatcacccagtaatacccatggtctccgtcacttccggggcatcttcaactgggtgctgagctatcggcgtgattcagatatctttgtaccctatggtcgcttagagcccttctctgggcctacgcccccactaccagccaaaagcaggatggctgcctgggtggtcagcaatttccaggagcggcagcagcgggcaaagctgtaccggcagctggcccctcatctgaaggtggatgtgttcggtcgcgccagtggacggcccctgtgccctaactgtctgctgcccactgtggcccggtaccgcttctacctgtcctttgagaactcacagcaccgggactacatcaccgagaagttctggcgcaatgcactggccgctggcgctgtgcctgtggtgctgggacctcctcggactacctacgaggctttcgtgccaccagatgcctttatacacgtagatgacttcagctctgcccgtgaactagctgtcttccttgtcagcatgaatgagagtcgctatcgtggcttctttgcttggcgagaccggctccgtgtgcggctcctgaatgactggagggagcgcttctgcaccatctgcgcccgctacccttacttgccccgcagccaggtctatgaagacctggaaagctggttccaagcttgaactcctgctgctgggagaggctgtgtgcgtggaagactgatgatgaaatggaagggcttttgggtcaccatgggactaaccctaggcttaggtcagtgaataggaagtcaggatatgaggagaaggctgggctgagaagccatatggatgaggactctggtgggctttagagtagggacccagggaaggagacaattaatgaggagcgtgtgggaaaagctagtcaaacggggaaagtggctgagggtcctggacttaccttgagtatgctcatggctcaaggctcaggtgaaaaagggaggcagtgtctctggagctgggaacatccaaagctgggatttgtggggacaaacatggtgcctgaacctccacaatcaaagtgcttagcctcagggatacaagtgtgtgttccagaactccacatgcaaaatgtatgctgagcccagggtttgtgggagaaggatggtgtggatgattctgggcttttgacaccacagttccctgagggaaagaggcaccactaataataaaattgttcacttgtaatagagacgccaaaaaaaaaaaaaaaaaaaaaaaaaaaagt

SEQ ID NO: 8 - DNA, 1190 bp, Helicobacter pylori
atgcctttgttgagagcaccaagctaccccccccccccccccacaaaaagcctttaaatttagacttacaatggtatcgcgatttaaatgattttaaggggtggtttttttatgacattttccagcaccaatacaatgtttcgtttgataaaaaggcggattgtgattgcctccttggagtgcaccatagaacactagaagagttgtaccaagccttgactaatcccaaaaagcgtcttgtgtttatgggagaaaatgagcgcattgattttaatgtctacgactttgccatgagttttgatcatttggagtttggagatcgctatttgcgcgtgcctctttattaccagtctctccattggttcttgcacatcattactcatgcgtctaatcctccttttagactggatggagcgcacgagatgctacactctcctttgacactccatctaccccttaactccacatgcactaaaatttcttttgctgacaaataccctcatttagatgccctagctagagaacagaaaaatcctcttgaacgagagtttgctagttttgtggcttccaactggggagcccccatgcgcaataatttttaccagcaactcaacgagtatcgccctgtggctgggggtgggagagtgtttaacactataggaaaacccgtatctaataaacatgactttttagcccaatataagttcaatttgtgctttgaaaactcttgtggcatgggttataccactgagaaaattgtggatgcttattttgcccacactattcctatttattggggtaatccgttagtgcatttggattttaaccctaaaagttttgtgaatgtccatgattttgccaacctagatgaggcgatcgattttgtgcactatttggacacccatgacaatgcctatttagaaatgctccatgctcacccgcttagcattgtggaaggcaaaccgaaattttgccatgatttgagttttaagaaaatcttagactttttggtcaatgcgatagaaagtccgcataactatcacgagcaggtccgtgtacttagtaacagtgtgtacaaatatcggcatccaagccgcgacaccgtgctagaagcatgctctggaagagaacatctgcaactcctcctcaaaaagctccataaaaaattatggaagcgcaagagaccctaa

SEQ ID NO: 9 - DNA, 28 bp, Unknown
aagaactcgagtcaaaagtcgctctcat

SEQ ID NO: 10 - DNA, 27 bp, Unknown
ttataagcttttatgactccagcgcga

SEQ ID NO: 11 - DNA, 32 bp, Unknown
caagaaagatctcaagtaaacaacgagttttt

SEQ ID NO: 12 - DNA, 28 bp, Unknown
ttatgagctcttacccccgaaagcggtc

SEQ ID NO: 13 - protein, 16 aa, Unknown
VDFSGGWSDTPPLAYE

SEQ ID NO: 14 - protein, 18 aa, Unknown
TGIRDWDLWDPDTPPTER

SEQ ID NO: 15 - protein, 12 aa, Unknown
LSWEQLQPCLDR

SEQ ID NO: 16 - DNA, 30 bp, Unknown
ccttaattaagagcagtcagagggaagtca

SEQ ID NO: 17 - DNA, 25 bp, Unknown
ccagggccttgctgattaattaagg

SEQ ID NO: 18 - DNA, 23 bp, Unknown
gagtattctagattggggcctga

SEQ ID NO: 19 - DNA, 21 bp, Unknown
ggaaaatgcgtgcagtccaca

SEQ ID NO: 20 - DNA, 33 bp, Unknown
ctaggcactgaagggaacaaagtgtcgatcctc

SEQ ID NO: 21 - DNA, 27 bp, Unknown
aggcgttgactgtagccaccggagtga

SEQ ID NO: 22 - DNA, 36 bp, Unknown
gactccaggcttcatgtgtaggggaaatccacgtac

SEQ ID NO: 23 - DNA, 34 bp, Unknown
cactgacagttcaatgtcatctgcacaggtgacc

SEQ ID NO: 24 - DNA, 28 bp, Unknown
ctctgtcctagcaaacactgctgtgccg

SEQ ID NO: 25 - DNA, 25 bp, Unknown
gaggaacctgagcctgtggactgca

SEQ ID NO: 26 - DNA, 23 bp, Unknown
ccgctcgaggagactctccgcga

SEQ ID NO: 27 - DNA, 32 bp, Unknown
ggggtaccttaagctagcatgtcttgtacatc

(19) United States
(12) Patent Application Publication
(10) Pub. No.: US 2002/0058313 A1
(43) Pub. Date: May 16, 2002

(54) Use of recombinant enzymes for preparing GDP-L-fucose and fucosylated glycans

(76) Inventors: Risto Renkonen, Espoo (FI); Pirkko Mattila, Espoo (FI); Laura Hirvas, Helsinki (FI); Solveing Hortling, Helsinki (FI); Tuula Kallioinen, Vantaa (FI); Sirkka-Liisa Kauranen, Espoo (FI); Nina Jarvinen, Saukkola (FI); Minna Maki, Helsinki (FI); Jaana Niittymaki, Espoo (FI); Jarkko Rabina, Helsinki (FI)

Correspondence Address: CROWELL & MORING LLP, INTELLECTUAL PROPERTY GROUP, P.O. BOX 14300, WASHINGTON, DC, 20044-4300, US

(21) Appl. No.: 09/962,805

(22) Filed: Sep. 26, 2001

(30) Foreign Application Priority Data
Sep. 26, 2000 (FI) 20002114

(51) Int. Cl. C12P 19/02; C12N 9/04; C12N 1/18

(52) U.S. Cl. 435/105; 435/254.2; 435/320.1; 435/190

(57) ABSTRACT

Use of recombinant enzymes for the preparation of GDP-L-fucose and fucosylated glycans is disclosed. GDP-L-fucose functions as a fucose donor in the biosynthetic route leading to the fucosylated glycans, which have therapeutic utility. A process for preparing GDP-L-fucose and fucosylated glycans, and means useful in the process are provided. Said means include enzymes, chimeric enzymes, DNA sequences, genes, vectors and host cells. An assay for the determination of GDP-fucose and fucosyltransferase, and a test kit therefore are also provided.

### FIELD OF THE INVENTION

The present invention relates to the use of recombinant enzymes for preparing GDP-L-fucose and fucosylated glycans. Fucosylated glycans are biologically active and have therapeutic utility, and GDP-L-fucose functions as a fucose donor in the biosynthetic route leading to the fucosylated glycans.

More precisely, the present invention is directed to a process for preparing GDP-L-fucose and fucosylated glycans, and to means useful in said process. Accordingly, the invention is also directed to DNA sequences and genes encoding useful enzymes and to the enzymes. Chimeric enzymes comprising several enzymes of the biosynthetic pathway are also provided. The invention further relates to vectors comprising the DNA sequences encoding the enzymes or chimeric enzymes, and to host cells comprising the vectors. Finally, the invention provides an assay useful in the process of the invention for the determination of GDP-fucose or fucosyltransferase, and a test kit therefore. Said assay may also be useful in the diagnosis of infection or inflammation.

### BACKGROUND OF THE INVENTION

Fucosylated glycans are useful in the treatment of inflammatory responses. They can be used to block leukocyte traffic to sites of inflammation and thus reduce or otherwise ameliorate an undesired inflammatory response and other disease states characterized by a leukocyte infiltrate. They are also useful in blocking bacterial adherence to endothelium and thus they prevent and/or treat bacterial infections. A further use of the fucosylated glycans lies in the field of cancer treatment where metastasis of tumor cells can be inhibited by these glycans (U.S. Pat. No. 5,965,544).

The migration of white blood cells from the blood to regions of pathogenic exposure in the body is called the inflammatory cascade. Cell adhesion events allow specific binding of a leukocyte to the endothelium of the vessel that is adjacent to the inflammatory insult; such adhesion events counteract the high vascular shear forces and high blood flow rates that tend to keep the leukocyte circulating, and help guide the leukocyte to the required site.

The current concept of leukocyte extravasation is based on the consecutive action of several adhesion molecules located on the surface of leukocytes and the endothelium. Lymphocyte extravasation is initiated by the interaction of members of the selectin family and their oligosaccharide-containing counterreceptors.

Selectins, also known as “lectin cell adhesion molecules” (LEC-CAMs), are classified into three groups: L-selectin is expressed on various leukocytes, and is constitutively expressed on lymphocytes, monocytes, neutrophils, and eosinophils. E-selectin is expressed on endothelium activated by inflammatory mediators. P-selectin is stored in alpha granules of platelets and Weibel-Palade bodies of endothelial cells and is also expressed on endothelium activated by inflammatory stimuli. All members of the selectin family appear to mediate cell adhesion through the recognition of carbohydrates.

All selectins bind to sialyl Lewis x (Neu-NAcα2-3Galβ1-4(Fucα1-3)GlcNAc) (sLe^{x} or sLex) and sialyl Lewis a (NeuNAcα2-3Galβ1-3(Fucα1-4)GlcNAc) (sLe^{a} or sLea) as well as to related carbohydrate sequences. L-selectin-dependent recognition precedes normal lymphocyte extravasation into peripheral lymph nodes and into sites of inflammation, both of which are impaired in L-selectin deficient mice.

Several glycoproteins have been shown to act as counterreceptors for L-selectin. A common nominator for the cloned ligands GlyCAM-1, CD34, MAdCAM-1 and PSGL-1 is the mucin type protein core rich in O-linked glycan decorations which are crucial for selectin recognition. The glycosylation of GlyCAM-1 and PSGL-1 has been characterized in greater detail, among other saccharides these proteins have been shown to carry sulfated sLex and sLexLexLex epitopes, respectively

High endothelial cells in peripheral lymph nodes express sialyl Lewis a and sialyl Lewis x (sLea and sLex) epitopes, which are parts of the L-selectin counter-receptor. The endothelial cells in several other locations are sLea and sLex negative, but inflammatory stimuli can induce previously negative endothelium to express these oligosaccharide structures de novo. It has been shown that cultured endothelial cells possess the machinery to generate at least sLex, since they have several functional α-2,3-sialyl- and α-1,3-fucosyltransferases, enzymes involved in generating sLex from (poly)lactosamines.

A number of studies have proposed that selectins are involved in a wide variety of acute and chronic inflammatory conditions in many tissues. Mono- and multivalent sLex glycans have been shown to inhibit L-selectin mediated lymphocyte binding in vitro and they also inhibit granulocyte extravasation in in vivo animal models of acute inflammation and reperfusion injury. Polylactosamines carrying single epitopes of sLexLex- and sLexLexLex-type appear to be recognized by E- and P-selectins with higher affinity than analogous oligosaccharides bearing single sLex-units. WO 97/12892 discloses synthetic multivalent sLex containing polylactosamines and their use to block lymphocyte binding to correspondent oligosaccharides on the endothelial surface.

Fucosylated glycans such as sLex and/or Lex, have been shown to be crucial in selectin dependent extravasation of leukocytes and tumor cells as well as in bacterial and parasitic infections (Vestweber D, and Blanks J E: Mechanisms that regulate the function of the selectins and their ligands. Physiol. Rev. 1999, 79:181-213). Fucosylation of glycans on glycoproteins and -lipids requires the enzymatic activity of relevant fucosyltransferases and GDP-L-fucose as the donor. Due to the biological importance of fucosylated glycans, a readily accessible source of GDP-L-fucose would be required. Currently GDP-L-fucose is still a relatively expensive nucleotide sugar and thus laboratories working in the field of fucosylation would benefit of more accessible sources of it.

There are two major strategies for the synthesis of GDP-L-fucose, the chemical and the enzymatical pathways. Various approaches have been used in the relatively complex chemical synthesis starting from L-fucose, the endproduct being GDP-L-fucose. Such syntheses are, however, very laborious, expensive and inconvenient for producing L-fucose on the large scale.

In eukaryotic cells GDP-L-fucose can be synthesized via two different pathways, either by the more prominent de novo pathway or by the minor salvage pathway. Procaryotes have only the de novo pathway. The predominant de novo route starts from GDP-D-mannose and the minor salvage pathway uses L-fucose as the starting material (Becker D J, and Lowe J B: Leukocyte adhesion deficiency type II (Review). Biochim. Biophys. Acta—Molecular Basis of Disease. 1999, 1455:193-204)

The first step of the de novo pathway starting from GDP-D-mannose is a dehydratation reaction catalyzed by a specific nucleotide-sugar dehydratase, GDP-mannose-4,6-dehydratase (GMD). This leads to the formation of an unstable GDP-4-keto-6-deoxy-D-mannose, which undergoes a subsequent 3,5 epimerization and to the formation of a GDP-4-keto-6-deoxy-D-galacatose, which then underogoes a NADPH-dependent reduction resulting in the formation of GDP-L-fucose (FIG. 1). These two last steps are catalyzed by a single, bifunctional enzyme GDP-4-keto-6-deoxy-D-mannose-3,5-epimerase/4-reductase (GFS), also called GDP-L-fucose synthetase. The genes coding for GMD and GFS activities for de novo pathway have been cloned from several bacteria, plants and mammals (Tonetti M, Sturla L, Bisso A, Benatti U, and De Flora A: Synthesis of GDP-L-fucose by the human FX protein. J. Biol. Chem. 1996, 271:27274-9).

The salvage pathways utilizes L-Fuc and converts it to GDP-Fuc via two enzymatic reactions (FIG. 1). The two enzymatic steps are catalyzed by fuco-1-kinase (FK) and GDP-fucose-pyrophosphorylase (PP). FK converts L-Fucose into L-Fucose-1-P and PP converts the L-Fucose-1-P into GDP-L-fucose. The salvage pathway has been successfully performed with purified enzymes in a recycling one-pot approach, but not yet with recombinant enzymes (Ichikawa Y, Look G C, and Wong C H: Enzyme-catalyzed oligosaccharide synthesis. Analytical Biochemistry 1992, 202:215-38).

L-fucose is a crucial monosaccharide present in several biologically important glycans (Feizi T, and Galustian C: Novel oligosaccharide ligands and ligand-processing pathways for the selecting. TIBS 1999, 24:369-372). In prokaryotes L-fucose is mainly present in polysaccharides of the cell wall and in animals L-fucose is a part in glycoconjugates, such as ABH-and Lewis antigens, either bound to the cell membrane or secreted into biological fluids. Fucosylation requires the action of fucosyltransferase, which catalyses the transfer of L-fucose from a nucleotide sugar, GDP-L-fucose, into the glycan acceptor.

The biosynthesis of GDP-L-fucose, which acts as a nucleotide-sugar donor for fucosylation, has received increased interest after a number of α-1,2-, α-1,3- and α-1,6-fucosyltransferase genes have been cloned, sequenced and expressed. Fucosyltransferases catalyze the transfer of fucose from a nucleotide sugar, GDP-L-fucose (GDP-Fuc), to a saccharide acceptor.

Some fucosyltransferases have been cloned from various sources and thus they have become available for synthetic purposes. However, GDP-Fuc is still a relatively expensive nucleotide sugar and therefore a more accessible source of it would be desirable. The availability and high costs limiting the large-scale oligosaccharide synthesis could be overcome by enzymatic synthesis of GDP-Fuc from cheaper carbohydrate resources.

One aim of the present invention is to increase the efficacy to synthesize GDP-L-Fuc. Another aim of the present is to facilitate the production of fucosylated glycans from GDP-L-Fuc. Fucosylated glycans have biologically useful properties, which can be utilized in glycobiology research and medicinal applications. The present invention thus provides processes and means for producing GDP-L-fucose and fucosylated glycans by recombinant gene technology.

Still another aim of the present invention is to provide an assay for the diagnosis of infection or inflammation.

### SUMMARY OF THE INVENTION

One object of the present invention is to provide a process for preparing GDP-L-fucose and fucosylated glycans, wherein said compounds are prepared using one or more recombinant enzymes from their biosynthetic routes. Another object of the invention is the use of recombinant enzymes for preparing GDP-L-fucose and fucosylated glycans.

Further objects of the invention are isolated DNA sequences encoding Helicobacter pylori GDP-mannose-4,6-dehydratase (GMD), Helicobacter pylori GDP-4-keto-6-deoxy-D-mannose-3,5-epimerase/4-reductase (GFS), murine or human fuco-1-kinase (FK) rat or murine GDP-fucose-pyrophosphorylase (PP) or Helicobacter felis or rat α-1,3-fucosyltransferase.

Still further objects of the invention are the isolated genes comprising the DNA sequences and the enzymes encoded by the sequences.

The invention further provides a chimeric enzyme, which is a chimeric molecule of GDP-mannose-4,6-dehydratase (GMD) and GDP-4-keto-6-deoxy-D-mannose-3,5-epimerase/4-reductase (GFS), or a chimeric molecule of fuco-1-kinase (FK) and GDP-fucose-pyrophosphorylase (PP).

Additional objects of the invention are vectors comprising one or more of the DNA sequences set forth above and double vectors comprising a first DNA sequence encoding GDP-mannose-4,6-dehydratase (GMD) and a second DNA sequence encoding GDP-4-keto-6-deoxy-D-mannose-3,5-epimerase/4-reductase (GFS), or a first DNA sequence encoding fuco-1-kinase (FK) and a second DNA sequence encoding GDP-fucose-pyrophosphorylase (PP). Host cells comprising the vectors are also provided.

Finally, the invention provides an assay for the determination of GDP-fucose, said assay comprising employing biotinylated carbohydrate-polyacrylamide conjugates, streptavidin and time-resolved fluorometric detection, and test kits comprising reagents needed for performing the assays.

Preferred embodiments of the invention are set forth in the dependent claims.

### BRIEF DESCRIPTION OF THE DRAWINGS

FIG. 1 shows two biosynthetic routes for GDP-L-fucose;

FIG. 2 shows the vector pESC-leu/gmd/wcaG;

FIG. 3 shows the vector pESC-trp/mFK; and

FIG. 4 shows the vector pESC-trp/PP.

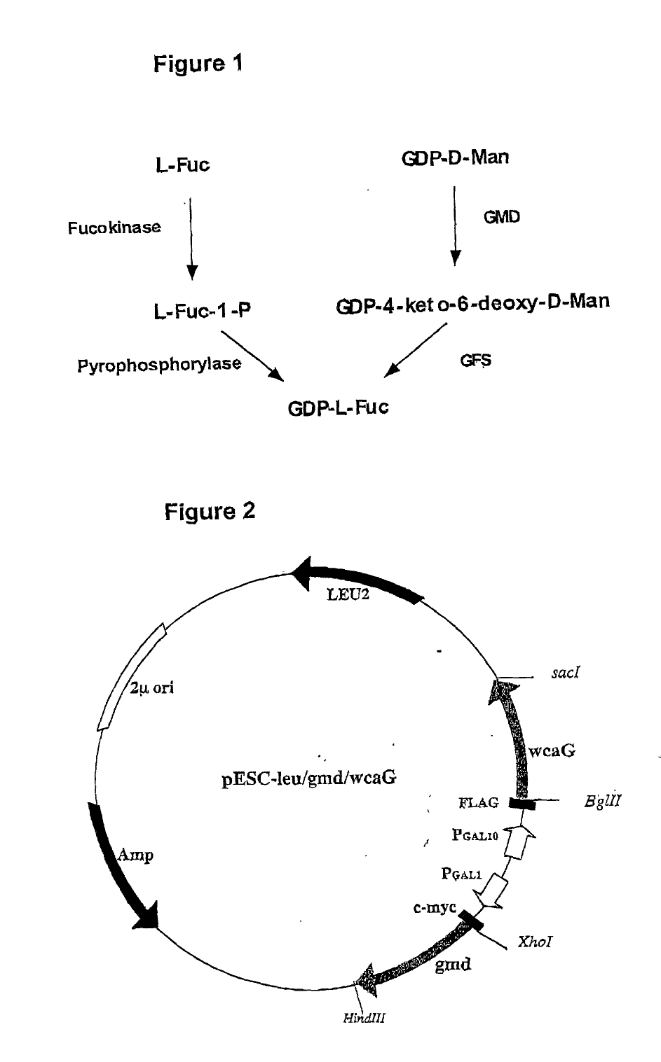

### DETAILED DESCRIPTION OF THE INVENTION

Abbreviations used: AAL, Aleuria aurantia lectin; FucT, α-1,3-fucosyltransferase; GDP-Fuc or GDP-L-Fuc, GDP-L-fucose; GDP-Man or GDP-D-Man, GDP-D-mannose; GFS, GDP-4-keto-6-deoxy-D-mannose-3,5-epimerase/4-reductase (GFS) or GDP-L-fucose synthetase; GMD, GDP-D-mannose dehydratase; Lex, Lewis x (Galβ1-4(Fucα1-3)GlcNAc); sLex, sialyl Lewis x (Neu5Acα2-3Galβ1-4(Fucα1-3)GlcNAc); sLN, sialyllactosamine (Neu5Acα2-3Galβ1-4GlcNAc).

GDP-L-fucose can be prepared using recombinant enzymes of either of the two known biosynthetic routes. In principle, the recombinant enzymes can be produced in any prokaryotic or eukaryotic cell that has been transformed with a vector comprising a sequence coding for GMD, GFS, FK or PP. GDP-L-Fuc can then be produced by reacting the appropriate substrate with the enzyme converting it. The enzyme may be in the form of a cell lysate, or it may be isolated therefrom and further purified. The two enzymes needed to convert GDP-Man or L-Fuc, respectively, into GDP-L-Fuc may even be produced in different organisms and separately added to the reaction mixture. Preferably, the host cell is yeast or mold, especially Saccharomyces cerevisiae. Yeast and mold cells contain intrinsic GDP-D-Man, which can serve as substrate for the biosynthesis of GDP-L-Fuc via the de novo pathway.

A very convenient way for the recombinant production of GDP-L-Fuc is to construct a double vector, i.e. a vector comprising the two genes needed for the de novo pathway or the two genes needed for the salvage pathway. A host cell transformed with such a double vector can produce a chimeric enzyme having the activity of both enzymes. A yeast or mold cell comprising both gmd and gfs genes can produce GDP-L-Fuc without external addition of GDP-D-Man.

GDP-L-fucose serves as fucose donor for the fucosylation of glycans by fucosyltranseferases. According to the present invention, fucosylated glycans are prepared by reacting GDP-L-Fuc with recombinantly produced α-1,3-fucosyltransferase, preferably obtained from rat or bacteria. The host cell can be any appropriate prokaryotic or eucaryotic cell, including animal cells.

The glycans to be fucosylated are preferably polylactoseamines, which are converted into sialyl Lewis x (sLex) sugars. The α-1,3-fucosyltransferase transfers fucose in α-1,3-position to a GlcNAc-sugar. GlcNAc can be part of the polylactoseamine backbone e.g. Galβl,4GlcNAcβb1,3-Galβ1,4GlcNAc, which in turn may be part of a glycoprotein or a glycolipid. In other words, the process of the present invention can be used for fucosylating sugars as such, including naturally occurring and synthetically produced ones, or for fucosylating glycoproteins and glycolipids. All these fucoglycosylated compounds have potential use as selectin inhibitors. They may be further formulated into pharmaceutical compositions.

The present invention provides a number of novel genes which are useful in the claimed process. Said genes comprise a DNA sequence encoding a certain enzyme from a certain organism. The sequenced DNA sequences are given as SEQ ID NO.S 1-8. The present invention is especially directed to the enzyme coding parts (starting at ATG) of these sequences. However, it is to be understood that the invention is not limited to the exact sequences, but it includes any sequences that encode the stated enzymes. The invention also includes the complementary sequences, hybridizing sequences and sequences which, but for the degeneracy of the genetic code, would hybridize to them.

The DNA sequences are incorporated into vectors together with appropriate promoters and selection markers. Suitable promoters are e.g. GAL promoters. Antibiotic resistance or essential amino acids may be used as selection markers. The vectors are transformed or transfected into host cells and the recombinant host cells are then cultivated under conditions allowing expression of the encoded enzymes. The enzymes are then recovered from the cells, or the cell lysate is used directly, to catalyze a desired enzymatic reaction. The enzyme or the cell lysate comprising the enzyme is then contacted with its substrate to form the desired product. Alternatively, the enzyme reaction is carried out within the recombinant host cell, which thus directly produces the desired reaction product.

Assays for the determination of GDP-Fuc or fucosyltransferase facilitate the monitoring of the process of the invention. The assays are especially suitable for the determination of GDP-L-Fuc, but the same principle may also be applied to the determination of other sugar nucleotides. Correspondingly, the determination of fucosyltransferases is especially suited for the determination of α-1,3-fucosyltransferase, but the same principle may also be applied to the determination of α-1,2- or α-1,6-fucosyltransferases. Since fucosylation increases during infection and inflammation, the assays of the present invention can also be used in diagnosis. Increased amounts of GDP-Fuc and/or fucosyltransferases in the body indicate infection or inflammation diseases. The GDP-Fuc and fucosyltransferase activity may be determined in any tissue sample. Since fucosyltransferase is also secreted, its activity can further be determined in any body fluid sample, such as blood serum, plasma, spinal cord fluid, joint fluid, tears, saliva etc. The assays may also be used in determining the presence of the gmd, gfs, fk or pp genes or their mRNA or the corresponding enzymes.

According to one embodiment of the invention, the assay comprises incubating a sample suspected to contain GDP-fucose with a fucosyltransferase and a sLN-polyacrylamide-biotin conjugate to form a biotinylated fucosylated glycoconjugate (sLex conjugate); contacting the reaction mixture of the previous step with immobilized streptavidin to immobilize the biotinylated sLex conjugate; reacting the biotinylated sLex conjugate with a primary anti-sLex antibody, and then with a secondary europium-labelled antibody that recognizes the primary antibody; and detecting any time-resolved fluorescence as a measure of GDP-fucose in the sample.

Alternatively, the assay comprises incubating a sample suspected to contain fucosyltransferase with GDP-L-fucose and a sLN-polyacrylamide-biotin conjugate to form a biotinylated fucosylated glycoconjugate (sLex conjugate) ; contacting the reaction mixture of the previous step with immobilized streptavidin to immobilize the biotinylated sLex conjugate; reacting the biotinylated sLex conjugate with a primary anti-sLex antibody, and then with a secondary europium-labeled antibody that recognizes the primary antibody; and detecting any time-resolved fluorescence as a measure of fucosyltransferase in the sample.

A test kit useful in the assay above can contain e.g. the fucosyltransferase or GDP-Fuc, the sLN-polyacrylamide-biotin conjugate, optionally immobilized streptavidin, the primary anti-sLex antibody, and the labelled secondary antibody, and optionally reaction and washing solutions.

According to another embodiment of the invention, the assay comprises immobilizing a biotinylated Lex-polyacrylamide conjugate onto a carrier coated with streptavidin; adding a sample suspected to contain GDP-fucose, and europium-labeled fucose-specific lectin AAL, incubating and washing; and detecting any time-resolved fluorescence, whereby a decrease in the fluorescence indicates the amount of GDP-Fuc present in the sample.

A test kit useful in this assay may comprise biotinylated Lex-polyacrylamide conjugate, optionally immobilized streptavidin and labeled fucose-specific lectin AAL. It may also comprise appropriate reaction and washing solutions.

GMD and GFS

Here we describe the molecular identification, cloning and expression strategies of identifying novel genes coding for this pathway and their coexpression in yeast cells such as S. cerevisiae. Novel gmd and gfs (also termed gmers, or fx) genes from H. pylori were identified, cloned, sequenced and functionally expressed together in a double-vector in S. cerevisiae. The corresponding E. coli genes can also be succesfully expressed in this yeast system. The great benefit of this yeast cell approach is the fact that the expression host has spontaneously very high levels of GDP-D-Mannose, the precursor of GDP-L-Fuc synthesis, in the cytosol and furthermore, it lacks primarily the genes and enzymes involved in the fucose metabolism.

Also two chimeric molecules, namely GMD-GFS and GFS-GMD, were generated by PCR and the enzymatic activity could be shown even with this approach. The great benefit of this approach is that this way the otherwise unstabile GMD is better preserved and furthermore, only a one-step purification of the enzyme(s) is needed before it can be coupled to a matrix, etc.

We have also probed whole-genome gene-chips of S. cerevisiae having either or both gmd and gfs genes transfected. By this approach we can identify the genome-wide expression patterns of genes and identify pathways that are disrupted due to the transfections. We are able to perform metabolic engineering to the yeast strains transfected with novel genes and further enhance the productivity of the GDP-L-Fuc.

Previous work has shown that gmd and gfs can be overexpressed in H. pylori and that when exogenous GDP-D-mannose is introduced into the reaction, GDP-L-fucose is synthesized. However, our work offers a major improvement to this approach as no exogenous expensive GDP-D-mannose needs to be added to the yeast cell lysates expressing GMD and GFS (GFS) enzymes.

The availability of GDP-D-mannose was shown to be a limiting factor for our yeast transformant expressing gmd as well as gfs genes. In yeast cells GDP-D-mannose is synthesized in the cytoplasm and further transported to the lumenal space of Golgi apparatus by a specific antiporter system that involves exchange with guanosine 5′-monophosphate (GMP). Thus, as the recombinant enzymes are expressed in the cytosolic compartment of the yeast, it might be beneficial to use mutant yeast cells, which have been characterized to have a defect in the GMP antiporter function leading to increased cytosolic GDP-D-mannose levels.

Taken together, we have shown here that bacterial gmd and gfs genes can be expressed as functional enzymes in S. cerevisiae and due to the inherent GDP-D-mannose synthesis in yeast cells they synthesize GDP-L-fucose. This approach was shown to be relatively effective as>0.2 mg/l GDP-L-fucose was produced by specifically transfected yeast cells. It should also be noted that no optimization of the yeast cell culture systems has been performed so far to increase the yield. The GDP-L-fucose generated by this rapid route can be further converted to bioactive fucosylated glycans with relevant recombinant fucosyltransferases.

FK and PP

The salvage pathway utilizes L-Fuc and converts it to GDP-L-Fuc via two enzymatic reactions catalyzed by fuco-1-kinase (FK) and GDP-L-fucosephosphorylase (PP). Here we describe the molecular identification, cloning and expression strategies of identifying novel genes coding for the first step of the salvage pathway, i.e. fuco-1-kinase. In this pathway fukokinase utilizes L-Fuc and conversts it to L-Fuc-1-P. We have first identified the FK enzyme by using the three known peptide sequences known from the enzyme and generating in silico probes from them. With these probes and with the help of both human and murine EST cDNA databanks, we were able to pull out the putative human and murine fk genes. The murine fk gene can then be expressed in S. cerevisiae and other cells, such as bacterial, yeast, insect and mammalian host by novel multiplatform vectors (such as gateway), and the enzymatic activity was demonstrated in a system which normally is devoid of background fucose metabolism. The tissue expression pattern of the novel fk gene was analysed by Northen blotting and relatively high levels of expression were detected in different organs.

The second enzyme in this pathway is the PP enzyme (GDP-L-fucose-pyrophosphorylase), which converts the Fuc-1-P into GDP-L-Fuc. The human PP (hPP) enzyme has previously been sequenced. Now we have sequenced the gene coding for murine and rat PP. Using sequence homology we were able to PCR the murine and rat pp's from the kidney cDNA libraries. We show that there are at least two variants of this gene in the mammalian genome and, after cloning and sequencing the pp gene, we have also expressed it in the S. cerevisiae. Preferably these two genes, fk and pp are expressed in a double vector in order to generate a chimeric molecule and thereby increase the efficacy to synthesize GDP-L-Fuc.

Transferases

α-1,3-Fucosyltransferase enzymes

Fucosyltransferase transfers GDP-L-Fuc (donor) to a growing glycan chain. We have cloned, sequenced and expressed a bacterial α-1,3-fucosyltransferase enzyme from H. felis. Furhermore, we have cloned, sequenced and expressed a novel rat α-1,3-fucosyltransferase (FucT-VII) enzyme, which is crucial in the synthesis of sialylated glycans. Both of these genes code enzymes that can be used to α-1,3-fucosylate glycan and glycoproteins with the help of GDP-L-fucose.

Assays

To this end, we have developed high-through put assays for the identification of GDP-L-Fuc in cell extracts. We have two assays, the first one detects smaller amounts of GDP-L-Fuc present in the sample and relays on the activy of a specific fucosyltransferase enzyme, and the newly synthetized glycans are detected with relevant monoclonal antibodies in a time-resolved immunofluorometric assay and the second relays on the specific detection of GDP-L-Fuc by fucose-binding activity of AAL lectin.

We have recently developed rapid and sensitive high-throughput assays for glycosyltransferases and glycosidases, which employ microplate assay technology and time-resolved fluorometric detection. In the detection of GDP-Fuc, we utilized α-1,3-fucosyltransferase assay, in which the limiting factor was the presence or absence of GDP-Fuc in the sample. The α-1,3-fucosyltransferase reaction converting sialyllactosamine (sLN) to sialyl Lewis x (sLex)-tetrasaccharide was performed in the solutionphase, after which the biotinylated glycoconjugate was immobilized onto streptavidin-coated microplate. The fucosylated reaction product sLex was then detected by time-resolved immunofluorometry. This assay combines the advantages of solution-phase enzymatic reaction and solid-phase detection technology, being versatile and capable of simultaneous processing of multiple samples.

A faster low-cost method based on inhibition of europium-labelled fucose-specific Aleuria aurantia lectin (AAL) was also developed for measuring GDP-Fuc concentration. The lectin-based assay is less sensitive than the enzymatic assay, but as a cheap and rapid method it is well-suited for optimizing production of GDP-Fuc in yeast. As a large variety of different lectins, glycosyltransferases, and glycoconjugates are commercially available, the newly developed methods are applicable to the analysis of many different nucleotide sugars and glycosyltransferases as well.

The use of conventional chromatographic and radiochemical methods in quantitating GDP-Fuc is complicated by the fact that different nucleotide sugars are similar in structure and the cellular concentration of GDP-Man is high in yeast. Furthermore, in optimizing the conditions for the production of GDP-Fuc, large amounts of samples have to be monitored. Thus, we took advantage of stereospecific recognition of fucose by either α-1,3-fucosyltransferase enzyme or a fucose-specific lectin, the functions of which are easy to detect in microplate assays. The advantages of the new assays include ease of procedure and capability of simultaneous processing of multiple samples.

In the enzyme assay, solution-phase enzymatic reaction and solid-phase detection were performed. In the FucT assay earlier published, we used immobilized acceptor, but later we have noticed that a soluble acceptor is clearly better for this enzyme. The reason for this is unknown, but probably it is kinetically advantageous that all components of the enzymatic reaction are in the solution-phase.

As an end point assay, FucT assay is more sensitive and precise in the determination of GDP-Fuc concentration than the lectin inhibition assay, in which the affinity of the lectin to fucose determines the sensitivity. Thus the benefits of time-resolved fluorometric detection are better seen in FucT assay. Time-resolved fluorometry is based on the unique fluorescence properties of some lanthanide chelates and has proved to provide remarkably high sensitivity and a wide range of measurements in noncompetitive assays. Compared to the enzymatic assay, the lectin inhibition assay works with higher concentrations of GDP-Fuc, and the concentrations of GDP-Fuc in the samples we used in this study were only slightly over detection limit. However, in optimizing the production of GDP-Fuc, better yields will probably be achieved and this method is a promising tool because the components of the assay are cheap and incubations take under 1 hour.

The two assays complement each other; the enzymatic assay is suitable for the detection of minute amounts of GDP-Fuc, whereas the robust and cheap lectin assay requires micromolar concentrations of GDP-Fuc before it can be used. Both assays work well with crude cell lysates, but if necessary, GDP-Fuc can be purified with Aleuria aurantia lectin affinity chromatography. The enzyme-based method can be used in measuring of both de novo and salvage reaction pathways, but the lectin-based method is applicable only with the de novo pathway, as free fucose would disturb the assay.

In conclusion, the methods we have developed should prove very useful in monitoring the production of GDP-Fuc and in the analysis of fucose metabolism in cells. These assays are attractive alternatives to the currently used methods especially in screening of large numbers of crude biological samples. The methods used in this study can also be easily adapted to the analysis of other enzymes in glycobiology.

The following examples illustrate the present invention.

### EXAMPLE 1

Functional expression of Escherichia coli enzymes synthesizing GDP-L-fucose from inherent GDP-D-mannose in Saccharomyces cerevisiae

Preparation of gene constructs

The gmd and wcaG coding regions were amplified from E. coli K-12 wca gene cluster (accession U38473) . Advantage polymerase (Clontech, Palo Alto, Calif., USA) was used with the primer set 5′AAGAACTCGAGTCAAAAGTCGCTCTCAT3′ (SEQ ID NO:9) (creating a XhoI-site) and 5′TTATAAGCTTTTATGACTCCAGCGCGA3′ (SEQ ID NO:10) (creating a Hind III-site) to amplify gmd (nucleotides 8662-9780) as well as primer set 5′CAAGAAAGATCTCAAGTAAACAACGAGTTTTT3′ (SEQ ID NO:11) (creating a Bgl II-site) and 5′TTATGAGCTCTTACCCCCGAAAGCGGTC3′ (SEQ ID NO:12) (creating a Sac I-site) to amplify wcaG (nucleotides 9786-10748). Both PCR-products were cloned into PCR-blunt II TOPO (Zero-blunt-TOPO; PCR-kloning kit, Invitrogen, Groningen, The Netherlands). Both inserts were digested out of PCR-blunt II TOPO and transferred to pESC-leu vector (Stratagene, La Jolla, Calif., USA). The gmd-gene was subcloned under P_{GAL}1-promoter inframe with c-myc-epitope and the wcaG-gene under P_{GAL}10-promoter inframe with FLAG-epitope. The construct of this vector is shown in FIG. 2. Also vectors containing only either of the inserts were prepared. The constructs were confirmed by sequencing (ABI 310, PE Biosystems, Fostercity Calif., USA).

Transformation into S. cerevisiae

The pESC-leu vectors not containing any genes, containing either gmd or wcaG or both of the genes were transformed into YPH 499 and YPH 501 yeast host strains by lithium acetate method following the instructions of the manufacturer (Stratagene). Transformants were selected using leucine dropout plates.

Selection of yeast transformants

Several transformants growing on leucine dropout plates were picked up and grown in 25 ml of dextrose containing selective synthetic dropout (SD) media overnight. The GAL1 and GAL10 promoters were repressed when transformed yeast cells were grown in dextrose containing SD media and induced when the cells are changed to grow in galactose containing SG media. In a typical experiment the yeast cell mass was increased by growing them in SD media overnight and the expression of transformed genes was induced for another 24 h by changing the carbon source of the media from dextrose to galactose. After centrifugation the yeast cells were grown another 24 h in the same volume in synthetic galactose containing dropout (SG) media and the OD₆₀₀ was measured. 1×10⁹ cells were spinned down, suspended in 0.5 ml of breaking buffer containing 1% TX-100 and 10% glycerol and the cells were lysed mechanically by vortexing with glass beads (⅓ volume). After centrifugation the cell lysates were subjected to protein analysis as well as to enzymatic activity assays. 25 μg of total protein was used in Western blots and 0.3-0.4 mg/ml in the activity assys.

Expression of E. coli gmd and wcaG in yeast

Expression of recombinant proteins was detected on the RNA and the protein level. Total RNA (15 mg) was extracted from double transformant yeast cells as well as from negative controls and broken mechanically with glass beads and subjected to Northern blot analysis as described before (Mattila P, Joutsjärvi V, Kaitera E, Majuri M, Niittymäki J, Maaheimo H, Renkonen R, and Makarow M: Targeting of active rat a2,3-sialyltransferase to the yeast cell wall by the aid of the hsp150Dcarrier. Towards the synthesis of sLex decorated L-selectin ligands. Glycobiology 1996, 6:851-859). The blots were probed with PCR products amplified from E. coli K-12 wca gene cluster. The expression of GMD and GFS (GFS) was studied in Western blot using the antibodies against c-myc and FLAG-epitopes, respectively. Chemiluminescence (ECL, Amersham) was used as a detection method according to manufacturer's instructions.

Determination of GDP-L-fucose synthesis

The presence of GDP-L-fucose was assayed by using yeast cell lysate as a source of GDP-L-fucose in a fucosyltransferase reaction converting sialyl-N-acetyllactosamine (sLN) to sLex. The reaction mixture included fucosyltransferase VI (FucTVI 25 μU, Calbiochem; San Diego, Calif.), yeast cell lysate diluted 1:20 as a fucose donor, sLN-polyacrylamide-biotin conjugate (Syntesome; Moscow, Russia) as a fucose acceptor, 50 mM MOPS-NaOH (pH 7.5), 6 mM MnCl₂, 0.5% Triton X-100, 0.1% BSA, and 1 mM ATP. After 1 h incubation in +37° C. 50 μl aliquots of the reaction mixtures were transferred to microtitration strips coated with streptavidin (Wallac; Turku, Finland) to immobilize the biotinylated glycoconjugate. The fucosylated reaction product sLex was then detected and quantified by time-resolved fluorometry (Wallac) using anti-sLex primary antibody KM-93 (Calbiochem) and europium-labelled (DELFIA Eu-labelling kit: Wallac) anti-mouse IgM secondary antibody (Sanbio; Uden, The Netherlands) as described before (Räbinä J, Smithers N, Britten C J, and Renkonen R: A Time-Resolved Immunofluorometric Method For the Measurement Of Sialyl Lewis X-Synthesizing Alpha-1,3-Fucosyltransferase Activity. Analytical Biochemistry 1997, 246:71-78).

Purification of GDP-L-fucose

For a large-scale GDP-L-fucose synthesis, one transformant containing both gmd and wcaG genes was grown 24 h in 750 ml of selective SD media and another 24 h in selective SG media. The cells were collected when OD₆₀₀ was 3.7 and the pellet was suspended in concentration of 2×10 ⁹ cells/ml (total volume 45 ml) of breaking buffer containing 1% TX-100 and 10% glycerol and lysed with glass beads. After vigorous 30 min vortexing the glass beads and cell debris were centrifuged and the clear lysate was filtrated through YM-10 centricon column (Millipore Corporation, Bedford, Mass., USA) o/n +4° C. according to the manufacturer's instructions.

GDP-L-fucose synthesized in yeast cells was then purified in two chromatographic steps. Lectin affinity chromatography was performed on a small column (diameter 0.5 cm) of agarose-bound Aleuria aurantia lectin (2 ml; Vector laboratories, Burlingame, Calif.) . The column was equilibrated with 10 mM HEPES buffer, pH 7.5, containing 0.15 M NaCl and 0.02% NaN₃. After application of yeast cell lysate (4 ml) into the column, the elution was performed with 4 ml of the equilibration buffer followed by 4 ml of the buffer containing 25 mM L-fucose. Fractions of 1 ml were collected and assayed for the presence of GDP-L-fucose.

For desalting GDP-L-fucose and removal of the haptenic sugar, size-exclusion HPLC on a Superdex Peptide HR 10/30 column (Pharmacia, Sweden) was used. The elution was performed at 1 ml/min using 50 mM NH₄HCO₃ and the effluent was monitored with a UV detector at 254 nm. The amount of GDP-L-fucose was calculated from peak areas by reference to external standard (GDP-L-fucose, Calbiochem).

Matrix-assisted laser desorption/ionization time-of-flight mass spectrometry

Maldi-TOF mass spectrometry was performed with a Biflex mass spectrometer (Bruker Daltonics, Germany) . Analysis was performed in the negative-ion linear delayed-extraction mode, using 2,4,6-trihydroxyacetophenone (THAP, Fluka Chemica) as the matrix as described. External calibration was performed with THAP matrix dimer and sialyl Lewis x beta-methylglycoside (Toronto Research Chemicals, Canada).

Results

Preparation of gmd and wcaG gene constructs and screening of S. cerevisiae transformants

In order to induce GDP-L-fucose synthesis in S. cerevisiae the E. coli gmd coding for GDP-D-mannose-4,6 dehydratase (GMD) activity and wcaG coding for GDP-4-keto-6-deoxy-D-mannose epimerase/reductase, i.e GFS (GFS) activity, were inserted into pESC-leu-vector under GAL1 and GAL10 promoters, respectively (FIG. 2). The gmd was in frame with the c-myc-epitope and the wcaG with the FLAG-epitope as revealed by DNA sequencing.

The vector controls without any genes, single constructs containing only either gmd or wcaG or double constructs containing both genes were transformed into two yeast host strains YPH 499 and YPH 501. Several transformants of both host strains capable of surviving and proliferating on selective leucine deficient drop-out plates were screened for expression of gmd and wcaG -genes on the RNA level as well as the corresponding enzymes, GMD and GFS, on the protein level.

Expression of gmd and wcaG in S. cerevisiae

In Northern blot analysis a 1.3 kb transcript was detected for gmd in both yeast strains YPH 499 and YPH 501 transformed with either only E. coli gmd or both gmd and wcaG. When the presence of the corresponding enzyme, GMD was assayed with the c-myc antibody in Western blots, a 48 kD protein band was detected in the single transfectants containing only the gmd gene or double transfectants containing both the gmd and wcaG genes. Concomitantly the mock transformants showed no signals in either Northern or Western blots.

Correspondingly, a 1.1 kb band for wcaG was present in the Northern blot analysis from both yeast strains YPH 499 and YPH 501 transformed with either only E. coli wcaG or both gmd and wcaG genes. The corresponding enzyme coded by wcaG was expressed as a 40 kD band as detected with anti-FLAG-antibody. Again, no relevant bands were detected in mock controls or with control antibodies.

Enzymatic activity of E. coli GMD and GFS in yeast

The intermediate products of the de novo pathway from GDP-D-mannose to GDP-L-fucose (GDP-4-keto-6-deoxy-D-mannose and GDP-4-keto-6-deoxy-L-galactose) are known to be labile and not feasible to monitor in a high throughput screening approach. Thus we used a microwell plate assay developed in our laboratory for measuring the α-1,3-fucosylation. In this assay biotinylated polyacrylamide conjugated sLN and recombinant α-1,3-fucosyltransferase VI were added to yeast cell lysates either not containing (mock- or only single transformants with either gmd or wcaG) or containing GDP-L-fucose, i.e. double transformants with both the gmd and wcaG genes. The readout of this assay was the turnover of sialyllactosamine to sLex detected by specific antibodies and time-resolved immunofluorometry. As the relevant acceptor and enzyme were always present in this assay, the only limiting factor in the generation of sLex from sLN was the presence or absence of GDP-L-fucose.

Both the mock-transfectants as well as the yeast cells transformed separately only with gmd or wcaG were unable to synthesize GDP-L-fucose resulting in background values in the enzymatic assay. When the lysates of gmd and wcaG expressing yeast clones were mixed together, the synthesis of sLex was increased from 0% (background level) to 10.7% (YPH 499) or to 16.6% (YPH 501) in a one-hour assay. To prove that these genes could be transformed into the same yeast cell host, gmd was expressed under GAL1 promoter and wcaG under GAL10 promoter within the same pESC-leu vector in either YPH 499 or YPH 501. These double transfectants were able to produce enough GDP-L-fucose to enable 3.4% (YPH 499) and 5.1% (YPH 501) of the sLN acceptor to be converted to sLex in a one-hour assay.

To analyse if the amount of the inherent, yeast produced GDP-mannose was a limiting factor for the GDP-L-fucose synthesis in the double transformed yeast strains, external GDP-D-mannose was added (0-500 μM) to the yeast cell lysates before incubation with relevant sLN acceptor and recombinant α-1,3-fucosyltransferase. Addition of external GDP-D-mannose increased the amount of GDP-L-fucose synthesis 3-fold, i.e. from 3.4% to 12.5% (YPH 499 ) or from 5.1% to 12.5% (YPH 501). We also showed that the addition of exogenous NAPDH did not further enhance the synthesis of GDP-L-fucose suggesting that the concentration of this cofactor in the yeast cell lysates does not represent a limiting factor in this assay.

Purification and MALDI-TOF MS of GDP-L-fucose

To further confirm the synthesis and to quantitate the amount of GDP-L-fucose produced in the gmd and wcaG expressing yeast cells, Aleuria aurantia lectin affinity chromatography was used to purify the product. As monitored with the enzymatic α-1,3-fucosyltransferase assay, most of the GDP-L-fucose bound to the lectin column and was eluted with addition of exogenous L-fucose. The peak fraction was further purified by size-exclusion HPLC, in which the retention times of both the purified product and commercial GDP-L-fucose was 16.2 min. As compared to external standard (GDP-L-fucose), the amount of GDP-L-fucose after purification was 3 mg per 1 ml of the original yeast cell lysate, corresponding to 0.2 mg/l of GDP-L-fucose in the original yeast cell culture.

This HPLC-purified product was then subjected to a MALDI-TOF MS analysis. The product purified from the double transfected yeast cells comigrating with the commercial GDP-L-fucose gave a single peak at m/z 588.04, (calculated m/z for (M−H)⁻ of GDP-L-fucose is 588.08). A single peak seen in the appropriate area in MALDI-TOF MS not only indicates that the product is GDP-L-fucose, but also shows that it was free of other nucleotide sugars.

### EXAMPLE 2

Identification, cloning and functional expression of novel gmd and gfs genes from Helicobacter pylori

Using in silico cloning we were able to identify the putative gmd and gfs genes from the whole genome of the published H. pylori J99 (Genbank accession number AE001443). By sequence alignment the putative gmd gene was identified as jhp0038 in J99 and hp0044 (SEQ ID No. 1) in 26695, also known as rfbD. Similarly the gfs gene was suggested to be the jhp0039 in J99, hp0045 (SEQ ID No. 2), i.e. nolK in 26695, which has been named as wbcJ (McGowan).

In order to induce GDP-L-fucose synthesis in S. cerevisiae, the H. pylori gmd coding for GDP-D-mannose-4,6 dehydratase (GMD) activity and gfs coding for GDP-4-keto-6-deoxy-D-mannose epimerase/reductase, i.e GFS activity, were inserted into pESC-leu-vector under GAL1 and GAL10 promoters, respectively. The gmd was in frame with the c-myc-epitope and the gfs with the FLAG-epitope as revealed by DNA sequencing. The vector construct was equivalent to that shown in FIG. 2.

Preparation of gene constructs

The gmd and gfs coding regions were amplified from an H. pylori gene cluster (accession U38473). Advantage polymerase (Clontech, Palo Alto, Calif., USA) was used with the primer set 5′AAGAACTCGAGTCAAAAGTCGCTCTCAT3′ (SEQ ID NO:9) (creating a XhoI-site) and 5′TTATAAGCTTTTATGACTCCAGCGCGA3′ (SEQ ID NO:10) (creating a Hind III-site) to amplify gmd (nucleotides 8662-9780) as well as primer set 5′CAAGAAAGATCTCAAGTAAACAACGAGTTTTT3′ (SEQ ID NO:11) (creating a Bgl II-site) and 5′TTATGAGCTCTTACCCCCGAAAGCGGTC3′ (SEQ ID NO:12) (creating a Sac I-site) to amplify gfs (nucleotides 9786-10748). Both PCR-products were cloned into PCR-blunt II TOPO (Zero-blunt-TOPO; PCR-kloning kit, Invitrogen, Groningen, The Netherlands). Both inserts were digested out of PCR-blunt II TOPO and transferred to pESC-leu vector (Stratagene, La Jolla, Calif., USA). The gmd-gene was subcloned under P_{GAL}1-promoter inframe with c-myc-epitope and the gfs-gene under P_{GAL}10-promoter inframe with FLAG-epitope. Also vectors containing only either of the inserts were prepared. The constructs were confirmed by sequencing (ABI 310, PE Biosystems, Fostercity Calif., USA).

The vector controls without any genes, single constructs containing only either gmd or gfs or double constructs containing both genes were transformed into two yeast host strains YPH 499 and YPH 501. Several transformants of both host strains capable of surviving and proliferating on selective leucine deficient drop-out plates were screened for expression of gmd and gfs-genes on the RNA level as well as the corresponding enzymes, GMD and GFS, on the protein level.

Expression of gmd and gfs in S. cerevisiae

In Northern blot analysis a 1.3 kb transcript was detected for gmd in both yeast strains YPH 499 and YPH 501 transformed with either only H. pylori gmd or both gmd and gfs. When the presence of the corresponding enzyme, GMD, was assayed with the c-myc antibody in Western blots, a 48 kD protein band was detected in the single transfectants containing only the gmd gene or double transfectants containing both the gmd and gfs genes. Concomitantly the mock transformants showed no signals in either Northern or Western blots.

Correspondingly, a 1.1 kb band for gfs was present in the Northern blot analysis from both yeast strains YPH 499 and YPH 501 transformed with either only E. coli gfs or both gmd and gfs genes. The corresponding enzyme coded by gfs was expressed as a 40 kD band as detected with anti-FLAG-antibody. Again, no relevant bands were detected in mock controls or with control antibodies.

Enzymatic activity of H. pylori GMD and GFS in yeast

The intermediate products of the de novo pathway from GDP-D-mannose to GDP-L-fucose (GDP-4-keto-6-deoxy-D-mannose and GDP-4-keto-6-deoxy-L-galactose) are known to be labile and not feasible to monitor in a high throughput screening approach. Thus we used a microwell plate assay developed in our laboratory for measuring the α-1,3-fucosylation. In this assay biotinylated polyacrylamide conjugated sLN and recombinant α-1,3-fucosyltransferase VI were added to yeast cell lysates either not containing (mock or only single transformants with either gmd or gfs) or containing GDP-L-fucose, i.e. double transformants with both the gmd and gfs genes. The readout of this assay was the turnover of sialyllactosamine to sLex detected by spesific antibodies and time-resolved immunofluorometry. As the relevant acceptor and enzyme were always present in this assay, the only limiting factor in the generation of sLex from sLN was the presence or absence of GDP-L-fucose.

Both the mock-transfectants as well as the yeast cells transformed separately only with gmd or gfs were unable to synthesize GDP-L-fucose resulting in background values in the enzymatic assay. When the lysates of gmd and gfs expressing yeast clones were mixed together, the synthesis of sLex was increased from 0% (background level) to over 15% in a one-hour assay. To prove that these genes could be transformed into the same yeast cell, host gmd was expressed under GALL promoter and gfs under GAL10 promoter within the same pESC-leu vector in either YPH 499 or YPH 501. These double transfectants were able to produce enough GDP-L-fucose to enable the sLN acceptor to be converted to sLex.

### EXAMPLE 3

Cloning and functional expression of murine and human L-fucokinase (FK)

In silico and in vitro cloning of L-fucokinase

L-fucokinase (FK) has previously been purified to apparent homogeneity from pig kidney cytosol by Park et. al. 1998. The purified porcine enzyme has been subjected to endo-Lys-C digestion, followed by peptide isolation and their amino acid sequencing. This approach has yielded three peptides, with the following sequences:

peptide 1; VDFSGGWSDTPPLAYE (SEQ ID NO:13);

peptide 2; (T) (G) IRDWDLWDPDTP (P) (T) ER (SEQ ID NO:14); and

peptide 3; LSWEQLQPCLDR (SEQ ID NO:15).

In silico parts of the present work included BLAST search in a HTGS database and electronic Northern blots in the mouse EST database. In vitro PCR reactions to clone the murine fk gene were performed with probes identified in silico to match both putative human and murine sequences. The constructs were confirmed by sequencing (ABI 310, PE Biosystems, Fostercity Calif., USA).

Expression of murine FK protein

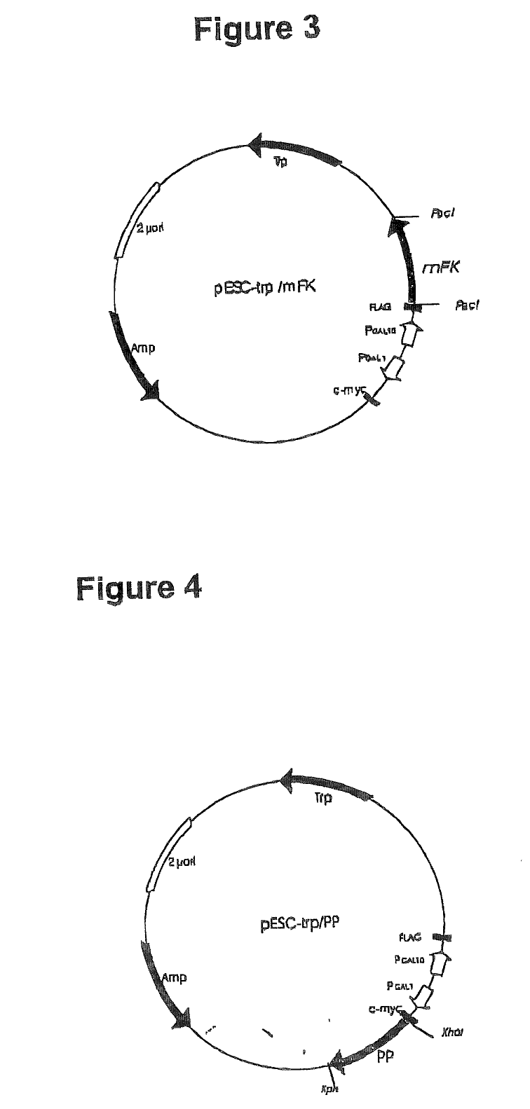

For protein expression the coding sequence of mouse FK (SEQ ID No. 3) nt 4-2388 was amplified with Advantage polymerase (Clontech, Palo Alto, Calif., USA) with the primer set SFKsc5′: 5′CCTTAATTAAGAGCAGTCAGAGGGAAGTCA3′ (SEQ ID NO:16) (creating the PacI site) and SFKsc3′:5′CCAGGGCCTTGCTGATTAATTAAGG3′ (SEQ ID NO:17) (creating the PacI site). PESC-trp vector was modified by digesting with BglII and SacI to remove the internal stop codon. The overhangs were filled with klenow polymerase and the vector was blunt end ligated into PCR-blunt II TOPO (Zero-blunt-TOPO; PCR-kloning kit, Invitrogen, Groningen, The Netherlands). The amplified fk sequence was inserted in PacI site and the orientation was verified by sequencing. The fk-gene was subcloned under P_{GAL}1-promoter under P_{GAL}10-promoter inframe with FLAG-epitope. The vector construction is shown in FIG. 3. The constructs were confirmed by sequencing (ABI 310, PE Biosystems, Fostercity Calif., USA).

Transformation into S. cerevisiae and selection of transformants

The pESC-leu vectors either containing or not containing the fk gene were transformed into YPH 499 and YPH 501 yeast host strains by lithium acetate method following the instructions of the manufacturer (Stratagene). Transformants were selected using leucine dropout plates.

Several transformants growing on leucine dropout plates were picked up and grown in 25 ml of dextrose containing selective synthetic dropout (SD) media overnight. The GAL10 promoters were repressed when transformed yeast cells were grown in dextrose containing SD media and induced when the cells were changed to grow in galactose containing SG media. In a typical experiment the yeast cell mass was increased by growing them in SD media overnight and the expression of transformed genes was induced for another 24 h by changing the carbon source of the media from dextrose to galactose. After centrifugation the yeast cells were grown another 24 h in the same volume in synthetic galactose containing dropout (SG) media and the OD₆₀₀ was measured. 1×10⁹cells were spinned down, suspended in 0.5 ml of breaking buffer containing 1% TX-100 and 10% glycerol and the cells were lysed mechanically by vortexing with glass beads (⅓ volume) . After centrifugation the cell lysates were subjected to protein analysis as well as to enzymatic activity assays.

Results

In silico and in vitro cloning of human and murine fk genes

On the basis of the previously published three peptide sequences we performed a Blast search in a HTGS database containing unfinished human high throughput genomic sequences. The putative human fk gene was found to be located in chromosome 16 (accession AC012184, Genbank). The genomic sequence (nucleotides 34031-64743) was analyzed in a gene structure prediction server (GENSCAN). The length of the corresponding putative human fk cDNA was 2469 bp (SEQ ID NO: 4), and the predicted corresponding FK protein of 823 amino acids.

When the predicted sequence of the human FK protein was used as a “probe” against mouse EST database, two EST sequences were found. The first EST sequence (accession number AI286399) had 91% identity on the nucleotide level with the 5′ and the other sequence (accession number AA509851) had 88% identity on the nucleotide level with the 3′end of the putative human sequence. This electronically identified putative murine fk sequence was then verified by PCR amplifying cDNA using the following primers: a 5′primer mFK 5′ (nucleotides 60-81 in AI286399) and 3′primer mfk stop (nucleotides 124-141 in AA509851).

The PCR reaction revealed a product of expected size and this was further sequenced.

Expression of the murine FK protein in yeast cells

The yeast cells (S. cerevisiase and Pichia pastoris) were selected as expression hosts because these cells are devoid of their own fucose metabolism and thus allow perfect experimental conditions. For protein expression the coding sequence of mouse fk (nt 4-2388) was amplified with the primer set SFKsc5′: 5′CCTTAATTAAGAGCAGTCAGAGGGAAGTCA3′ (SEQ ID NO:16) (creating the PacI site) and SFKsc3′:5′CCAGGGCCTTGCTGATTAATTAAGG3′ (SEQ ID NO:17) (creating the PacI site). PESC-trp vector was modified by digesting with BglII and SacI to remove the internal stop codon. The overhangs were filled with klenow polymerase and the vector was blunt end ligated. The amplified fk sequence was inserted in PacI site and the orientation was verified by sequencing.

The modified pESC-trp vector with the FLAG-epitope containing or not containing the mouse fk gene (FIG. 3) was transformed into S. cerevisiae strains YPH499 and YPH501. Positive clones were selected by Western blot (predicted weight of mFK is 87.5 kD) using anti-FLAG-antibody.

In summary this example describes the cloning and stable expression of a murine fk gene catalysing the reaction converting L-fucose to L-fucose-1-P. Using the information of three short peptide sequences, which were previously characterized from the purified porcine FK protein, we were able to electronically clone the human fk gene (2469 bp) and it was found to be located in chromosome 16 (accession AC012184) . The predicted human FK protein consisted of 823 amino acids. When the predicted sequence of the human FK protein was used as a “probe” against mouse EST database, two EST sequences were found. The first EST sequence (accession number AI286399) had 91% identity on the nucleotide level with the 5′ and the other sequence (accession number AA509851) had 88% identity on the nucleotide level with the 3′end of the putative human sequence. This electronically identified putative murine fk sequence was then verified by PCR amplifying cDNA using the following primers: a 5′primer mFK 5′ (nucleotides 60-81 in AI286399) and 3′primer mfk stop (nucleotides 124-141 in AA509851). This fk gene was present in several organs. When expressed in a S. cerevisiae or P. pastoris cell, devoid of background fucosylation we could identify an active enzyme. This enzymatic function was inhibited totally by excess of free fucose, 10% by excess of free arabinose while other monosaccharides had no effect.

### EXAMPLE 4

Cloning and expression of mouse and rat GDP-L-fucose pyrophosphorylase (PP)

Cloning strategy for the GDP-L-fucose pyrophosphorylase

For the in silico and in vitro cloning of the murine and rat GDP-L-fucose pyrophosphorylase genes, the human 3144 bp cDNA coding GDP-L-fucose pyrophosphorylase (hpp, Pastuszak et. al accession number 017445 in GenBank) sequence was used as a probe in the murine and rat EST libraries. A perfectly matching probe for screening cDNA rat and murine kidney cDNA libraries was made after alignments and used as follows: MF2 (5′GAGTATTCTAGATTGGGGCCTGA3′nucleotides 37-59 in AA422658) (SEQ ID NO:18) as a forward primer and MR (5′GGAAAATGCGTGCAGTCCACA3′ nucleotides 340-361 in AA422658) (SEQ ID NO:19) as a reverse primer. The PCR products were subcloned and subjected to sequencing. One clone was obtained from the murine library and three incomplete clones from the rat library. The missing 5′ends were obtained by RACE-PCR using gene specific primers:

RACE 1 (5′CTAGGCACTGAAGGGAACAAAGTGTCGATCCTC3′) (SEQ ID NO:20);

RACE 2 (5′AGGCGTTGACTGTAGCCACCGGAGTGA3′) (SEQ ID NO:21);

RACE 3 (5′GACTCCAGGCTTCATGTGTAGGGGAAATCCACGTAC3′) (SEQ ID NO:22); and

RACE 4 (5′CACTGACAGTTCAATGTCATCTGCACAGGTGACC3′) (SEQ ID NO:23).

cDNA alignments of mouse, rat and human pp sequences were performed. The mouse and rat sequences had SEQ ID NO:5 and SEQ ID NO:6, respectively.

Cloning the shorter transcript by 3′end RACE-PCR

3′end RACE PCR with two gene spesific primers MRrace1 (5′CTCTGTCCTAGCAAACACTGCTGTGCCG3′) (SEQ ID NO:24) and MRrace2 (5'GAGGAACCTGAGCCTGTGGACTGCA3') (SEQ ID NO:25) and mouse and rat kidney cDNA libraries as template as well as Southern blot were performed to prove the excistence of two transcripts from a single gene.

Expression of the mouse PP

For protein expression the coding sequence of mpp (nt 70-1842 of SEQ ID NO:5) was amplified with the primer set rmPPLH5U 5′CCGCTCGAGGAGACTCTCCGCGA3′ (SEQ ID NO:26) (creating the XhoI site) and rmPPLHlyh 5′GGGGTACCTTAAGCTAGCATGTCTTGTACATC3′ (SEQ ID NO:27) (creating the KpnI site) and ligated to the S. cerevisiae expression vector pESC-trp (FIG. 4).

pESC-trp vector was modified by digesting with BglII and SacI to remove the internal stop codon. The amplified pp sequence was inserted in PacI site and the orientation was verified by sequencing. The pp-gene was subcloned under P_{GAL}1-promoter inframe with c-myc-epitope. The constructs were confirmed by sequencing (ABI 310, PE Biosystems, Fostercity Calif., USA). The pESC-leu vector constructs either containing or not containign mpp gene were transformed into YPH 499 and YPH 501 yeast host strains by lithium acetate method following the instructions of the manufacturer (Stratagene). Transformants were selected using leucine dropout plates. Positive clones were selected by PCR showing the properly transfected cDNA clones and Western blot using anti-c-myc-antibody showing a protein of predicted weight of mouse/rat PP is 66 kDa.

Several transformants growing on leucine dropout plates were picked up and grown in 25 ml of dextrose containing selective synthetic dropout (SD) media overnight. The GAL10 promoters were repressed when transformed yeast cells were grown in dextrose containing SD media and induced when the cells were changed to grow in galactose containing SG media. In a typical experiment the yeast cell mass was increased by growing them in SD media overnight and the expression of transformed genes was induced for another 24 h by chancing the carbon source of the media from dextrose to galactose. After centrifugation the yeast cells were grown another 24 h in the same volume in synthetic galactose containing dropout (SG) media and the OD₆₀₀ was measured. 1×10⁹cells were spinned down, suspended in 0.5 ml of breaking buffer containing 1% TX-100 and 10% glycerol and the cells were lysed mechanically by vortexing with glass beads (⅓ volume). After centrifugation the cell lysates were subjected to protein analysis as well as to enzymatic activity assays.

Northern analysis and RT-PCR

The expression of pp transcripts in different rat and mouse tissues were analysed by Northern blot and RT-PCR. The Northern blot analysis revealed not only the expected 3.5 kb band reported also with the hPP, but also an additional band of 1.7 kb. Because the expression level of pp was very low in certain tissues, the Northern results were verified by RT-PCR. On the basis of these experiments we concluded that rPP was expressed in every tissue of the panel, although at low levels.

Enzymatic activity of PP

Biotinylated Lex- and sLN-polyacrylamide glycoconjugates were from Syntesome (Moscow, Russia) . Anti-sLex antibody KM-93 (mouse IgM) a1,3-fucosyltransferase VI (FucTVI) and GDP-Fuc were from Calbiochem (San Diego, Calif. Anti-mouse IgM (rat) antibody (Sanbio, Uden, The Netherlands) and streptavidin microtitration strips, enhancement solution, assay buffer, and wash concentrate were purchased from Wallac (Turku, Finland).

Preparation of yeast cell extracts

Recombinant yeast strains transformed or not transformed with the mpp gene were first grown overnight in glucose-containing SD-media and then the expression of the foreing genes was stimulated by growing the recombinant strains in galactose containing SG-media for 24 h. 1×10⁹ cells were spinned down, suspended in 0.5 ml of lysing buffer containing 50 mM MOPS-NaOH (pH 7.0), 1% Triton X-100, and 10% glycerol and the cells were lysed mechanically by vortexing with glass beads.

Measurement of GDP-Fuc with α-1,3-fucosyltransferase assay

The reaction mixture included the above mentioned yeast cells lysate containing the putative mPP enzyme, L-Fuc-1-P, sLN- polyacrylamide-biotin conjugate (1 μg/ml) as a fucose acceptor, fucosyltransferase (FucTVI 25 μU), 50 mM MOPS-NaOH (pH 7.5), 6 mM MnCl₂, 0.25% Triton X-100, 0.1% BSA, and 1 mM ATP. The reaction mixtures were incubated 1 h at +37° C. on ultra low binding 96-plates (Costar, Cambridge, Mass.). After enzymatic reaction, aliquots of 50 μl of the reaction mixtures were transferred to microtitration strips coated with streptavidin to immobilize the biotinylated glycoconjugates. After immobilization (30 min), the fucosylated reaction product sLex was detected and quantified by time-resolved fluorometry (Wallac) and monoclonal antibodies. The anti-sLex primary antibody KM-93 (0.5 μg/ml) and europium-labelled anti-mouse IgM secondary antibody (0.6 μg/ml) were both diluted with DELFIA assay buffer and incubated 1 h at room temperature with vigorous shaking. Between and after incubations, the strips were washed six times with DELFIA wash solution. Finally, DELFIA enhancement solution (150 μl/well) was added and incubated 5 min at room temperature with slow shaking. The fluorescence was then measured with a time-resolved fluorometer (Wallac). For quantitation of GDP-Fuc in samples, defined concentrations of commercial GDP-Fuc diluted with sample buffer were used as standards.

Results

In silico and in vitro cloning of murin and rat pp genes

On the basis of the previously published data on the 3144 bp long human GDP-L-fucose pyrophosphorylase (hpp) cDNA sequence (Pastuszak I, Ketchum C, Hermanson G, Sjoberg E J, Drake R, and Elbein A D: GDP-L-fucose pyrophosphorylase. Purification, cDNA cloning, and properties of the enzyme. J. Biol. Chem. 1998, 273:30165-74, accession number 017445 in GenBank) a 361 bp unknown mouse EST sequence (AA422658) was found, having 80% homology with the human sequence between nucleotides 1257-1621. A perfectly matching pair of primers for screening rat and murine cDNA kidney cDNA libraries was designed by alignment of the murine and human genes.

Detecting the enzymatic activity of mPP

We used our novel high-through put assay based on time-resolved immunofluorometry to detect the enzymatic activity of mPP in properly transfected yeast cell lysates. The expression of the mpp gene was stimulated by growing the recombinant strain in galactose containing SG-media for 24 h and lysed mechanically by vortexing with glass beads. The reaction mixture including the above mentioned yeast cells lysate containing the putative mPP enzyme was then incubated in the presence of L-Fuc-1-P and then sLN-polyacrylamide-biotin conjugate as a fucose acceptor and recombinant fucosyltransferase FucTVI were added for 1 hour. After enzymatic reaction, streptavidin immobilized the biotinylated glycoconjugates and the fucosylated reaction product sLex was then detected and quantified by time-resolved fluorometry with anti sLex monoclonal antibody. For quantitation of GDP-Fuc in samples, defined concentrations of commercial GDP-Fuc diluted with sample buffer were used as standards.

Our results show that the transformed yeast strains contain PP activity.

### EXAMPLE 5

Cloning of novel H. felis and rat α-1,3-fucosyltransferase genes

Fucosyltransferase transfers GDP-L-Fuc (donor) to a growing glycan chain. We have pulled out with low stringency cloning a bacterial α-1,3-fucosyltransferase enzyme from H. felis, cloned, sequenced and expressed it in E. coli, S. cerevisiae and Nawalma cells. The overall sequence homology and the conserved motif identifies it as a α-1,3-fucosyltransferase. The H. felis 1,3-fucosyltransferase gene has SEQ ID No. 8. Furhermore, we have also pulled out a novel rat α-1,3-fucosyltransferase (FucT-VII) enzyme, which is crucial in the synthesis of sialylated glycans, and cloned, sequenced and expressed in Nawalma cells.

The rat α-1,3-fucosyltransferase VII gene was cloned using cDNA from rat endothelial cells and primers designed based on homologies between human and murine genes. The cloned gene is 1910 bp long (SEQ ID No. 7) and the ORF is between nucleotides 194-1321 coding a 376 long amino acid. This gene was transfected to an expression vector pCDNA3 (with a neomycin resistance gene) and transfected to Namalwa cells by electroporation in order to establish a stable transfectant cell line with neomycin selection.

COS cells were transfected with the expression vector pCDNA3.1 (Invitrogen Inc. USA) comprising the rat FucT-VII gene or not comprising said gene (control). Fucosyltransferase activity was measured using sialyl lactosamine SA-alpha-2, 3Gal-beta-1, 4GlcNAc as acceptor whereby alpha-1,3-fucosylated sialyl Lewis x was formed. The specific fucosyltransferase activity of the COS/pCDNA3.1 vector was 0 pmol/mg/h and that of COS/pCDNA3.1+rat FucT-VII was 87 pmol/mg/h. The results confirm that the cloned rat FucT-VII codes for alpha-1,3-fucosyltransferase.

### EXAMPLE 6

Two time-resolved fluorometric high-throughput assays for quantitation of GDP-L-fucose from cell lysates, tissue extracts, and body fluids

Materials and methods

Biotinylated Lex- and sLN-polyacrylamide glycoconjugates were from Syntesome (Moscow, Russia) . Anti-sLex antibody KM-93 (mouse IgM), α-1,3-fucosyltransferase VI (FucTVI) and GDP-Fuc were from Calbiochem (San Diego, Calif.) and fucose-specific lectin AAL was from Vector Laboratories (Burlingame, Calif.). Anti-mouse IgM (rat) antibody (Sanbio, Uden, The Netherlands) and AAL were labelled with europium according to the manual of the DELFIA Eu-labelling kit (Wallac, Turku, Finland). Streptavidin microtitration strips, enhancement solution, assay buffer, and wash concentrate were purchased from Wallac.

Preparation of Yeast Cell Extracts

Recombinant yeast strains transformed with either GMD, GFS, or both of these genes were first grown overnight in glucose-containing SD-media and then the expression of the foreign genes was stimulated by growing the recombinant strains in galactose containing SG-media for 24 h. 1×10⁹ cells were spinned down, suspended in 0.5 ml of lysing buffer containing 50 mM MOPS-NaOH (pH 7.0), 1% Triton X-100, and 10% glycerol and the cells were lysed mechanically by vortexing with glass beads. The lysates and the mixture of single transformant lysates were incubated 2 h at +30° C.

α-1,3-Fucosyltransferase Assay

The reaction mixture included fucosyltransferase (FucTVI 25 μU), a fucose donor (commercial GDP-Fuc or yeast cell lysate), sLN-polyacrylamide-biotin conjugate (1 μg/ml) as a fucose acceptor, 50 mM MOPS-NaOH (pH 7.5), 6 mM MnCl₂, 0.25% Triton X-100, 0.1% BSA, and 1 mM ATP. The reaction mixtures were incubated 1 h at +37° C. on ultra low binding 96-plates (Costar, Cambridge, Mass.). After enzymatic reaction, aliquots of 50 μl of the reaction mixtures were transferred to microtitration strips coated with streptavidin to immobilize the biotinylated glycoconjugates. After immobilization (30 min), the fucosylated reaction product sLex was then detected and quantified by time-resolved fluorometry (Wallac) and monoclonal antibodies. The anti-sLex primary antibody KM-93 (0.5 μg/ml) and europium-labelled anti-mouse IgM secondary antibody (0.6 μg/ml) were both diluted with DELFIA assay buffer and incubated 1 h at room temperature with vigorous shaking. Between and after incubations, the strips were washed six times with DELFIA wash solution. Finally, DELFIA enhancement solution (150 μl/well) was added and incubated 5 min at room temperature with slow shaking. The fluorescence was then measured with a time-resolved fluorometer (Wallac).

Measurement of GDP-Fuc with a1,3-Fucosyltransferase Assay

Yeast cell lysate was used as a fucose donor in a fucosyltransferase reaction converting sLN to sLex (above). The samples were diluted (1:50) with 50 mM MOPS-NaOH buffer (pH 7.5) containing 0.5% Triton X-100 and mixed with other components of enzymatic reaction. For quantitation of GDP-Fuc in samples, defined concentrations of commercial GDP-Fuc diluted with sample buffer were used as standards.

Measurement of GDP-Fuc with Lectin Inhibition Assay

Lex-polyacrylamide-biotin (0.1 μg/ml in DELFIA assay buffer) was immobilized onto microtitration strips coated with streptavidin. After 30 min incubation and washing, either standards (GDP-Fuc diluted with lysing buffer) or yeast cell lysates (10 μl) were pipetted to wells. Eu-labelled AAL (40 μl, 0.2 μg/ml in DELFIA assay buffer) was then added. After 15 min incubation at room temperature with shaking, the strips were washed six times with DELFIA wash solution. Enhancement solution (150 μl/well) was added and the strips were incubated 5 min at room temperature with slow shaking. The fluorescence was then measured with a time-resolved fluorometer (Wallac).

Results

Two high-throughput microplate assays were developed for the quantitation of GDP-Fuc. Both assays employ biotinylated carbohydrate-polyacrylamide conjugates and time-resolved fluorometric detection. In the first assay (FIG. 2A) GDP-Fuc is used as a fucose donor for FucT enzyme, which transfers fucose to sLN producing sLex. The enzymatic fucosylation reaction proceeds in solution, after which the biotinylated glycoconjugates are specifically captured onto a microtitration plate coated with streptavidin. As constant concentrations of the enzyme and the acceptor are always present in this assay, the concentration of GDP-Fuc in the sample is the limiting factor in the generation of sLex from sLN. The immobilized reaction product sLex is detected with a product-specific primary antibody and europium-labelled secondary antibody. The second assay (FIG. 2B) is based on the binding of europium-labelled fucose-specific lectin AAL to Lexglycoconjugate immobilized onto microtitration plate wells. GDP-Fuc in the sample binds to the lectin and thus inhibits the binding of the lectin to the immobilized ligand, resulting in a decline in the fluorescence counts.

Standard Curves for GDP-Fuc

Defined concentrations of commercial GDP-Fuc were used as standards in the assays. In the first assay employing the enzymatic activity of FucT, the fluorescence counts were proportional to the concentration of GDP-Fuc in the range of 10-10 000 nM. The assay was capable of detecting 10 nM GDP-Fuc using enzyme incubation time of 1 h. Background counts were on the level of 10 000 cps and were subtracted from the results. In the second assay, relying on binding of the lectin to either matrix-bound ligand or GDP-Fuc in solution, GDP-Fuc inhibited the binding of AAL to the wells in a dose dependent way. When the binding data was converted to percentages, the inhibition concentration 50% (IC₅₀) was between 50-100 μM. The dynamic range of measurement is narrower than in the enzyme assay, being about 10-500 μM. In the low and high concentration ends of the standard curve the dose-response curve is shallow, which limits the sensitivity of the assay in these concentration areas.

Assays with Crude Yeast Lysates

The stable recombinant S. cerevisiae strain expressing the E. coli genes for GMD and GFS needed to convert GDP-Man to GDP-Fuc was tested together with single transformants and controls. As determined with the first, enzyme-based assay, the mock-transformant strain (501−) was unable to synthesize GDP-Fuc, resulting in background fluorescence values. In contrast, the double transformed strain (501+) was able to produce GDP-Fuc from its own GDP-Man pool. The strains expressing either GMD or GFS (single transformants) were unable to produce GDP-Fuc when tested alone. When the lysates of these strains were mixed together, the presence of GDP-Fuc was detected. The same samples were measured also with the second, lectin-binding assay. This assay is less sensitive, but as cold be predicted from the standard curves and the results of the enzyme-based assay, the presence of GDP-Fuc cold also be detected with this lectin assay. By using the standard curves, concentrations of GDP-Fuc in the cell lysates were calculated. In the lysate of double transformant yeast, the results given by the enzymatic and the lectin assay were 11 μM or 12 μM GDP-Fuc, respectively. Correspondingly, either 17 μM or 25 μM GDP-Fuc was detected in the mixture of the single transformant yeast lysates.

Although the foregoing refers to particularly preferred embodiments, it will be understood that the present invention is not limited to these. It will occur to those ordinarily skilled in the art that various modifications may be made to the disclosed embodiments and that such modifications are intended to be within the scope of the present invention.

All references mentioned herein are incorporated by reference in the disclosure.

We claim:

1. A process for preparing GDP-L-fucose from GDP-D-mannose, wherein the GDP-L-fucose is prepared from inherent GDP-D-mannose by cultivating yeast or mold cells, which have been transformed with a DNA sequence coding for GDP-mannose-4,6-dehydratase (GMD) and a DNA sequence coding for GDP-4-keto-6-deoxy-D-mannose-3,5-epimerase/4-reductase (GFS) to functionally express said enzymes, and by recovering the GDP-L-fucose formed from the GDP-D-mannose inherent in said cells.

2. The process of claim 1, wherein the GMD and the GFS expressed are enzymes of Escherichia coli or Helicobacter pylori.

3. The process of claim 1, comprising transforming yeast cells with a vector comprising said DNA sequences incorporated into a single vector.

4. The process of claim 1, comprising transforming the cells with a vector which encodes a chimeric molecule of GMD and GFS.

5. The process of claim 1, wherein the yeast cell is Saccharomyces cerevisiae.

6. The process of claim 1, further comprising preparing fucosylated glycans from the GDP-L-fucose formed.

7. The process of claim 6, wherein the fucosylated glycans are prepared from GDP-L-fucose by recombinant rat or bacterial α-1,3-fucosyltransferase.

8. The process of claim 6, wherein the fucosylated glycans are prepared by Helicobacter felis α-1,3-fucosyltransferase.

9. The process of claim 1, wherein the cells have been transformed with a DNA sequence encoding Helicobacter pylori GDP-mannose-4,6-dehydratase (GMD).

10. The process of claim 1, wherein the cells have been transformed with a DNA sequence encoding Helicobacter pylori GDP-4-keto-6-deoxy-D-mannose-3,5-epimerase/4-reductase (GFS).

11. The process of claim 9, wherein the cells have been transformed with a DNA sequence encoding Helicobacter pylori GDP-4-keto-6-deoxy-D-mannose-3,5-epimerase/4-reductase (GFS).

12. A chimeric enzyme which is a chimeric molecule of GDP-mannose-4,6-dehydratase (GMD) and GDP-4-keto-6-deoxy-D-mannose-3,5-epimerase/4-reductase (GFS).

13. A vector comprising a DNA sequence encoding a chimeric molecule of GDP-mannose-4,6-dehydratase (GMD) and GDP-4-keto-6-deoxy-D-mannose-3,5-epimerase/4-reductase (GFS).

14. A yeast or mold cell comprising the vector of claim 12.

15. A process for preparing GDP-L-fucose, wherein said compound is prepared from GDP-D-mannose through the de novo pathway using at least one of the recombinant enzymes selected from the group consisting of GDP-mannose-4,6-dehydratase (GMD) of Helicobacter pylori and GDP-4-keto-6-deoxy-D-mannose-3,5-epimerase/4-reductase (GFS) of Helicobacter pylori.

16. The process of claim 14, further comprising preparing fucosylated glycans from the GDP-L-fucose formed.

17. The process of claim 15, wherein the fucosylated glycans are prepared from GDP-L-fucose by recombinant α-1,3-fucosyltransferase, preferably by rat or bacterial α-1,3-fucosyltransferase.

18. An isolated DNA sequence encoding Helicobacter pylori GDP-mannose-4,6-dehydratase (GMD).

19. An isolated DNA sequence encoding Helicobacter pylori GDP-4-keto-6-deoxy-D-mannose-3,5-epimerase/4-reductase (GFS).

20. An enzyme which is encoded by the DNA sequence of claim 18.

21. An enzyme which is encoded by the DNA sequence of claim 19.

22. A vector comprising one or more of the DNA sequences of claim 18.

23. A vector comprising one or more of the DNA sequences of claim 19.

24. A host cell comprising the vector of claim 22.

25. A host cell comprising the vector of claim 23.

26. A process for preparing GDP fucose, wherein said compound is prepared from L-fucose through the salvage pathway using at least one of the recombinant enzymes selected from the group consisting of fuco-1-kinase (FK) and GDP-fucose-pyrophosphorylase (PP).

27. The process of claim 26, comprising transforming host cells with a vector comprising a DNA sequence encoding fuco-1-kinase (FK) and a DNA sequence encoding GDP-fucose-pyrophosphorylase (PP) to obtain transformed host cells that coexpress FK and PP, cultivating said cells, and recovering the GDP fucose formed.

28. The process of claim 1, further comprising preparing fucosylated glycans from the GDP fucose formed.

29. An isolated DNA sequence encoding murine or human fuco kinase (FK).

30. An isolated DNA sequence encoding rat or murine GDP fucose pyrophosphorylase (PP).

31. An isolated DNA sequence encoding Helicobacter felis or rat α-1,3-fu-cosyltransferase.

32. An enzyme which is encoded by the DNA sequence of claim 29.

33. An enzyme which is encoded by the DNA sequence of claim 30.

34. An enzyme which is encoded by the DNA sequence of claim 31.

35. A vector comprising the DNA sequence of claim 29.

36. A vector comprising the DNA sequence of claim 30.

37. A vector comprising the DNA sequence of claim 31.

38. A host comprising the vector of claim 29.

39. A host comprising the vector of claim 30.

40. A host comprising the vector of claim 31.

41. An assay for the determination of GDP Fucose or fucosyltransferase said assay comprising employing biotinylated carbohydratepolyacrylamide conjugates, streptavidin and time resolved fluorometric detection.

42. The assay of claim 41, comprising
incubating a sample suspected to contain GDP fucose or fucosyltransferase, respectively, with a fucosyletransferase or with GDP fucose, respectively, and a sLN polyacrylamide biotin conjugate to form a biotinylated fucosylated glycoconjugate (sLex conjugate)
contacting the reaction mixture of the previous step with immobilized streptavidin to immobilize the biotinylated sLex conjugate,
reacting the biotinylated sLex conjugate with a primary anti-sLex antibody, and then with a secondary europium labelled antibody that recognizes the primary antibody, and
detecting any time resolved fluoresence as a measure of GDP fucose or fucosyltransferase, respectively, in the sample.

43. The assay of claim 41, comprising
immobilizing a biotinylated Lex polyacrylamide conjugate onto a carrier coated with streptavidin,
adding a sample suspected to contain GDP fucose, and europiumlabelled fucose specific lectin ML, incubating and washing, and
detecting any time resolved fluorescence, whereby a decrease in the fluorescence indicates the amount of GDP Fuc present in the sample.

44. Test kit comprising reagents needed for performing the assay of claim 41.
